SEQUENCE LISTING (123 sequences)

SEQ ID NO: 1 - DNA, 20 bp, Mus musculus
  source 1..20: mol_type = genomic DNA
gttgatccgtttattccatt

SEQ ID NO: 2 - DNA, 20 bp, Mus musculus
  source 1..20: mol_type = genomic DNA
gtagaagtaaagcacgcgcc

SEQ ID NO: 3 - DNA, 20 bp, Homo sapiens
  source 1..20: mol_type = genomic DNA
gtgctccaaaacagtctgga

SEQ ID NO: 4 - DNA, 20 bp, Homo sapiens
  source 1..20: mol_type = genomic DNA
tcatagatgacaactttatc

SEQ ID NO: 5 - DNA, 21 bp, Mus musculus
  source 1..21: mol_type = genomic DNA
gttgatccgtttattccattc

SEQ ID NO: 6 - DNA, 22 bp, Mus musculus
  source 1..22: mol_type = genomic DNA
gttgatccgtttattccattct

SEQ ID NO: 7 - DNA, 23 bp, Mus musculus
  source 1..23: mol_type = genomic DNA
gttgatccgtttattccattcta

SEQ ID NO: 8 - DNA, 24 bp, Mus musculus
  source 1..24: mol_type = genomic DNA
gttgatccgtttattccattctac

SEQ ID NO: 9 - DNA, 25 bp, Mus musculus
  source 1..25: mol_type = genomic DNA
gttgatccgtttattccattctaca

SEQ ID NO: 10 - DNA, 21 bp, Mus musculus
  source 1..21: mol_type = genomic DNA
gtagaagtaaagcacgcgcct

SEQ ID NO: 11 - DNA, 22 bp, Mus musculus
  source 1..22: mol_type = genomic DNA
gtagaagtaaagcacgcgcctg

SEQ ID NO: 12 - DNA, 23 bp, Mus musculus
  source 1..23: mol_type = genomic DNA
gtagaagtaaagcacgcgcctga

SEQ ID NO: 13 - DNA, 24 bp, Mus musculus
  source 1..24: mol_type = genomic DNA
gtagaagtaaagcacgcgcctgag

SEQ ID NO: 14 - DNA, 25 bp, Mus musculus
  source 1..25: mol_type = genomic DNA
gtagaagtaaagcacgcgcctgagg

SEQ ID NO: 15 - DNA, 21 bp, Homo sapiens
  source 1..21: mol_type = genomic DNA
caaagctcagattggtcctcc

SEQ ID NO: 16 - DNA, 22 bp, Homo sapiens
  source 1..22: mol_type = genomic DNA
caaagctcagattggtcctcca

SEQ ID NO: 17 - DNA, 23 bp, Homo sapiens
  source 1..23: mol_type = genomic DNA
caaagctcagattggtcctccag

SEQ ID NO: 18 - DNA, 24 bp, Homo sapiens
  source 1..24: mol_type = genomic DNA
caaagctcagattggtcctccaga

SEQ ID NO: 19 - DNA, 25 bp, Homo sapiens
  source 1..25: mol_type = genomic DNA
caaagctcagattggtcctccagaa

SEQ ID NO: 20 - DNA, 21 bp, Homo sapiens
  source 1..21: mol_type = genomic DNA
gtgctccaaaacagtctggaa

SEQ ID NO: 21 - DNA, 22 bp, Homo sapiens
  source 1..22: mol_type = genomic DNA
gtgctccaaaacagtctggaaa

SEQ ID NO: 22 - DNA, 23 bp, Homo sapiens
  source 1..23: mol_type = genomic DNA
gtgctccaaaacagtctggaaac

SEQ ID NO: 23 - DNA, 24 bp, Homo sapiens
  source 1..24: mol_type = genomic DNA
gtgctccaaaacagtctggaaaca

SEQ ID NO: 24 - DNA, 25 bp, Homo sapiens
  source 1..25: mol_type = genomic DNA
gtgctccaaaacagtctggaaacac

SEQ ID NO: 25 - RNA, 20 nt, synthetic construct
  source 1..20: mol_type = other RNA
gttgatccgtttattccatt

SEQ ID NO: 26 - RNA, 20 nt, synthetic construct
  source 1..20: mol_type = other RNA
aatggaataaacggatcaac

SEQ ID NO: 27 - RNA, 20 nt, synthetic construct
  source 1..20: mol_type = other RNA
gtagaagtaaagcacgcgcc

SEQ ID NO: 28 - RNA, 20 nt, synthetic construct
  source 1..20: mol_type = other RNA
ggcgcgtgctttacttctac

SEQ ID NO: 29 - RNA, 20 nt, synthetic construct
  source 1..20: mol_type = other RNA
caaagctcagattggtcctc

SEQ ID NO: 30 - RNA, 20 nt, synthetic construct
  source 1..20: mol_type = other RNA
gaggaccaatctgagctttg

SEQ ID NO: 31 - RNA, 20 nt, synthetic construct
  source 1..20: mol_type = other RNA
gtgctccaaaacagtctgga

SEQ ID NO: 32 - RNA, 20 nt, synthetic construct
  source 1..20: mol_type = other RNA
tccagactgttttggagcac

SEQ ID NO: 33 - RNA, 21 nt, synthetic construct
  source 1..21: mol_type = other RNA
gaatggaataaacggatcaac

SEQ ID NO: 34 - RNA, 22 nt, synthetic construct
  source 1..22: mol_type = other RNA
gttgatccgtttattccattct

SEQ ID NO: 35 - RNA, 22 nt, synthetic construct
  source 1..22: mol_type = other RNA
agaatggaataaacggatcaac

SEQ ID NO: 36 - RNA, 23 nt, synthetic construct
  source 1..23: mol_type = other RNA
gttgatccgtttattccattcta

SEQ ID NO: 37 - RNA, 23 nt, synthetic construct
  source 1..23: mol_type = other RNA
tagaatggaataaacggatcaac

SEQ ID NO: 38 - RNA, 24 nt, synthetic construct
  source 1..24: mol_type = other RNA
gttgatccgtttattccattctac

SEQ ID NO: 39 - RNA, 24 nt, synthetic construct
  source 1..24: mol_type = other RNA
gtagaatggaataaacggatcaac

SEQ ID NO: 40 - RNA, 25 nt, synthetic construct
  source 1..25: mol_type = other RNA
gttgatccgtttattccattctaca

SEQ ID NO: 41 - RNA, 25 nt, synthetic construct
  source 1..25: mol_type = other RNA
tgtagaatggaataaacggatcaac

SEQ ID NO: 42 - RNA, 21 nt, synthetic construct
  source 1..21: mol_type = other RNA
gtagaagtaaagcacgcgcct

SEQ ID NO: 43 - RNA, 21 nt, synthetic construct
  source 1..21: mol_type = other RNA
aggcgcgtgctttacttctac

SEQ ID NO: 44 - RNA, 22 nt, synthetic construct
  source 1..22: mol_type = other RNA
gtagaagtaaagcacgcgcctg

SEQ ID NO: 45 - RNA, 22 nt, synthetic construct
  source 1..22: mol_type = other RNA
caggcgcgtgctttacttctac

SEQ ID NO: 46 - RNA, 23 nt, synthetic construct
  source 1..23: mol_type = other RNA
gtagaagtaaagcacgcgcctga

SEQ ID NO: 47 - RNA, 23 nt, synthetic construct
  source 1..23: mol_type = other RNA
tcaggcgcgtgctttacttctac

SEQ ID NO: 48 - RNA, 24 nt, synthetic construct
  source 1..24: mol_type = other RNA
gtagaagtaaagcacgcgcctgag

SEQ ID NO: 49 - RNA, 24 nt, synthetic construct
  source 1..24: mol_type = other RNA
ctcaggcgcgtgctttacttctac

SEQ ID NO: 50 - RNA, 25 nt, synthetic construct
  source 1..25: mol_type = other RNA
gtagaagtaaagcacgcgcctgagg

SEQ ID NO: 51 - RNA, 25 nt, synthetic construct
  source 1..25: mol_type = other RNA
cctcaggcgcgtgctttacttctac

SEQ ID NO: 52 - RNA, 21 nt, synthetic construct
  source 1..21: mol_type = other RNA
caaagctcagattggtcctcc

SEQ ID NO: 53 - RNA, 21 nt, synthetic construct
  source 1..21: mol_type = other RNA
ggaggaccaatctgagctttg

SEQ ID NO: 54 - RNA, 22 nt, synthetic construct
  source 1..22: mol_type = other RNA
caaagctcagattggtcctcca

SEQ ID NO: 55 - RNA, 22 nt, synthetic construct
  source 1..22: mol_type = other RNA
tggaggaccaatctgagctttg

SEQ ID NO: 56 - RNA, 23 nt, synthetic construct
  source 1..23: mol_type = other RNA
caaagctcagattggtcctccag

SEQ ID NO: 57 - RNA, 23 nt, synthetic construct
  source 1..23: mol_type = other RNA
ctggaggaccaatctgagctttg

SEQ ID NO: 58 - RNA, 24 nt, synthetic construct
  source 1..24: mol_type = other RNA
caaagctcagattggtcctccaga

SEQ ID NO: 59 - RNA, 24 nt, synthetic construct
  source 1..24: mol_type = other RNA
tctggaggaccaatctgagctttg

SEQ ID NO: 60 - RNA, 25 nt, synthetic construct
  source 1..25: mol_type = other RNA
caaagctcagattggtcctccagaa

SEQ ID NO: 61 - RNA, 25 nt, synthetic construct
  source 1..25: mol_type = other RNA
ttctggaggaccaatctgagctttg

SEQ ID NO: 62 - RNA, 21 nt, synthetic construct
  source 1..21: mol_type = other RNA
gtgctccaaaacagtctggaa

SEQ ID NO: 63 - RNA, 21 nt, synthetic construct
  source 1..21: mol_type = other RNA
ttccagactgttttggagcac

SEQ ID NO: 64 - RNA, 22 nt, synthetic construct
  source 1..22: mol_type = other RNA
gtgctccaaaacagtctggaaa

SEQ ID NO: 65 - RNA, 22 nt, synthetic construct
  source 1..22: mol_type = other RNA
tttccagactgttttggagcac

SEQ ID NO: 66 - RNA, 23 nt, synthetic construct
  source 1..23: mol_type = other RNA
gtgctccaaaacagtctggaaac

SEQ ID NO: 67 - RNA, 23 nt, synthetic construct
  source 1..23: mol_type = other RNA
gtttccagactgttttggagcac

SEQ ID NO: 68 - RNA, 24 nt, synthetic construct
  source 1..24: mol_type = other RNA
gtgctccaaaacagtctggaaaca

SEQ ID NO: 69 - RNA, 24 nt, synthetic construct
  source 1..24: mol_type = other RNA
tgtttccagactgttttggagcac

SEQ ID NO: 70 - RNA, 25 nt, synthetic construct
  source 1..25: mol_type = other RNA
gtgctccaaaacagtctggaaacac

SEQ ID NO: 71 - RNA, 25 nt, synthetic construct
  source 1..25: mol_type = other RNA
gtgtttccagactgttttggagcac

SEQ ID NO: 72 - RNA, 21 nt, synthetic construct
  source 1..21: mol_type = other RNA
  misc_feature 21: note = 3' dTdT
gttgatccgtttattccattc

SEQ ID NO: 73 - RNA, 21 nt, synthetic construct
  source 1..21: mol_type = other RNA
  misc_feature 21: note = 3' dTdT
gaatggaataaacggatcaac

SEQ ID NO: 74 - RNA, 21 nt, synthetic construct
  source 1..21: mol_type = other RNA
  misc_feature 21: note = 3' dTdT
gtagaagtaaagcacgcgcct

SEQ ID NO: 75 - RNA, 21 nt, synthetic construct
  source 1..21: mol_type = other RNA
  misc_feature 21: note = 3' dTdT
aggcgcgtgctttacttctac

SEQ ID NO: 76 - RNA, 21 nt, synthetic construct
  source 1..21: mol_type = other RNA
  misc_feature 21: note = 3' dTdT
caaagctcagattggtcctcc

SEQ ID NO: 77 - RNA, 21 nt, synthetic construct
  source 1..21: mol_type = other RNA
  misc_feature 21: note = 3' dTdT
ggaggaccaatctgagctttg

SEQ ID NO: 78 - RNA, 21 nt, synthetic construct
  source 1..21: mol_type = other RNA
  misc_feature 21: note = 3' dTdT
caaagctcagattggtcctcc

SEQ ID NO: 79 - RNA, 21 nt, synthetic construct
  source 1..21: mol_type = other RNA
  misc_feature 21: note = 3' dTdT
ggaggaccaatctgagctttg

SEQ ID NO: 80 - RNA, 21 nt, synthetic construct
  source 1..21: mol_type = other RNA
gttgatccgtttattccattc

SEQ ID NO: 81 - DNA, 7025 bp, Mus musculus
  source 1..7025: mol_type = genomic DNA
ggaaggggcggggcgctggagggggcggggcgtcgcggagggcctagctgcccagaggtagtctccagctccgcggtgctgctgaggagaaggaggagaatgtgagccgccgcccggcctcccaagacgatgctcgctgtcgtgggcgcggcggccctggtgctggtggccggggcgccttgggtgctaccctcagctgcaggtggagaaaatctgaaacctcctgagaatatagacgtctacattatagatgacaactacaccctaaagtggagcagccacggagagtcaatgggcagtgtgaccttttcagcagaatatcgaacaaaagacgaggcgaagtggttaaaagtgcctgaatgtcaacatactacaacgaccaagtgtgaattctctttactggacacaaatgtgtatatcaaaacacagtttcgtgtcagagcagaggaagggaacagcacatcttcgtggaatgaggttgatccgtttattccattctacacagctcacatgagccccccagaagtacgtttagaagctgaagataaagccatactagtccacatctctcctcccggacaagacgggaacatgtgggcactggagaaaccttccttcagttacaccatacgaatctggcagaagtcttccagtgacaaaaaaactattaactctacgtattatgtagaaaagataccagaactcttgccagagactacttactgtttagaagttaaagcaatacatccgtcacttaagaaacacagcaattacagcactgtgcagtgtataagcaccacagtggcaaataaaatgcctgtgccaggaaatctccaagtggatgcccaaggcaagagctatgtcctgaaatgggactacattgcgtctgcagacgtgctcttcagggcacagtggcttcctggctattcaaaaagcagttctggaagccgttcagataaatggaaaccaataccaacctgtgcaaatgtccagactacgcactgtgtcttttctcaagatactgtctacacaggaacgttctttctccatgtacaagcctcagagggaaatcacacatccttttggtctgaagagaagtttattgattctcaaaaacacattctccctcctcctccggtcattactgtcaccgccatgagtgacaccttgcttgtttatgtcaactgtcaggacagcacatgtgatggactcaattacgaaatcatcttttgggaaaacacttccaatactaagataagcatggagaaggatggcccagagttcaccctcaagaacctgcagccgctgactgtgtactgtgtccaggccagagtgctcttcagggccctgctgaataagaccagcaacttcagtgaaaagctgtgtgagaaaacacgtccaggaagtttttccacgatctggattataactggattaggtgttgtgttcttctctgtcatggtcctttatgctttgaggagcgtctggaaatacctgtgtcatgtgtgcttcccaccactcaagcctccccgcagtattgatgagtttttctctgagccgccttcaaaaaaccttgtacttctgacggctgaggagcacacggaaagatgcttcatcattgagaatacagacacggtcgctgtagaagtaaagcacgcgcctgaggaggacctcaggaagtacagctcacagaccagccaggactcgggcaactattccaacgaagaggaggagagtgtgggcaccgagagcggccaagctgtgctctccaaagctccctgcggggggccatgcagcgtgcctagccctcctgggaccttggaagacgggacctgcttcctgggaaatgaaaaatatcttcagagcccagccctgaggacagagccagctcttctctgctgacaggtgctcggcccaggctgggggccgtgtggaggagacagacttatctgaagtggctgccagcttctgctgccagctggaagccctgggtgctgtttgtgccacagatgaggctcactgatgtcaggcatctggtggttctgtctcggtgtttgtcctctaggtgacatttagaacattccgatttttaatcctgccgtagcccctcagtgtgtcacaggtaccattgtctacagccgcgcttcctaacccatacctcaccacagccgtggcttctgaccagaagtcactgaaaccatggagaaaggggacactaactggaggagctggcgttgtttaagatctcagtgtggcctgggtgtgcccgttgtactctagtacttgcagaactagaaaatgccagggacaaaaagcaggcatgaaccattcagtaagagagaagctagtgaacctgatggtgaaagaagaccgtgtccagtcagtgtttgtgtgaaggccctgtgctgctctctgtagccttcccgtggtacttataaaaccaggccgaaccctgtcctctgtccggcactattcaggtcccttcccagctccaggtacttaaaaaacaaagtgttgggtcctggggtgagggaacagcttcgacggcacagtgctgcctgcaggcgcctataagtgaccttagatgaatgctccatgagcatcttcggtaggcaggacatccagcagtgctggtggctccagggcagactcagttgcaggccaaaaaggaagagagaggagtcatagccctgccccaatcgagcttgttgctcgctgcactgcgcctttgtaatcaatcttggtttttttgttttgttttgttttttttaacttttaggtgataacatattctcagatatagcacacaagagacactacacaaattacgtctgaaagttttgatagaaaagagcttttgactggggtagacagcttgctcctcactactaccttgtatctcagtctcctcaacagagagagaatgcagattgattttcatcctgagttttattttcttgcccggtttctgggttcgggtgtgctttcaaggctctgaacagcttgtctgcccacaggggagcccggagacccagccttttggctaaatgttgcaggttcctgtacttgaaggactccagctcctctgtagagggcatggtcatcgcggtctgcaatcacgctgatacccagaaaaatggccacacaactcaaatctggtgccacgctttaataattctacccagcacggcccagccctgttcacggcggaaaagctccttctgccaggacagcctgtgaagaaacaagacctccgaggacgtggccaaggaaggcccagatgctagcgggaagttgttagttggcactctttcatctctcaacaactgtgacacaggtcttcagggtgaagcccaaagcagctatcggttgcagcagcgggtaaggagggggcttcacctgagcctggccttctgggcgagcagttctacctgcacacttcaagacagcctccatgacccttttcacggcacaccgcctgcttttggtgtgaccggggtttcctgtgcaggcacatgttcttaaggaatgccatgcacacaggcctgaagagctggctcagcgggtagagcacaggttgctcttgtctgggatctgagctctgtccccagcacccacagagtgcttctggttgcctcagactccaggggtgcacaaggccatgtaagcaaagacatgtaagcaaagacagttgtacacagagaagtcaaacctttttgaaaaaaagaaacaccacgggctggagagatggctcagtggttaagaacactgaccactcttccagaggtcctgagttcaattcccagcaaccacatggtggctcacaaccatctgtaataggatctgatgcccccttctggtgtgtctgaagatagctacagtgtattcatatataaaataataaatacatccttttttttctttttttactattggatattttctttatttacatttcaaatgttatcccctttcctgatttacccccctcccagaagccccctatcccatcccccctcccctgcttctatgagggtgttcacccacccacccactcccacttccctgcccttgattcccctacactgggttatttatcaagccatcataggaccaaagacctctccttccattgatccatgacaaagccatcctctgcaacatatgcagcttgagctatgtgtacttcttggttgatggcatagtccctgggagttctggggcagtggggggttggggggagtctggttagttgatattattcttcctattggggttgcaaaccccttcaatgccttcagtcccttctctaactcctctattagggaccccgagctcagtccagtggttggctgctaacatccacctctgtatttgtaaggctctggcagggcctctcaagagacagccatatcaggctcctttcagcatgtacttcttggcacccacaacagtgtctggtttggtaacgctgtacggcatgaattccgaggtgggacaatctatgggtggcctttcattcagtctctgctctacactttatctccatatttgctccagtgagtattttgttctccttctaagaaggacagaagcacccacactttgctcgtccttcttattgagcttcatgtggtctgtgaattgtatcctggttatttggagctttggggctagtatccacttatcagtgagtacatattgtgtgtgttcttttgtgattgggttacctcactcaggatgatatcctccagatccatccatttgcctaagaatttcatgaattcatcgtttttaatagctgtgcagtactctgttgtggaaatgcaccacgttttctgtatccattcctctgttgaggggcatctgggttctttccagcttctggctattagaaataaggctgctatgaacatagtggagcatgtgtccttcttaccagttggagcatcttctgggtatatgcccaggagtagcatagctgggtcctcaggtaatactatgtccaattttctgaggaccggccaaactgatttccagagtggttgtacgagcttgtattcccaccagcaatggaggagtgttcctctttctccacatcctcaccaacatctgctgtcaacagagtttttaattttagccattctgactggtgtgaaatggaatctcagggtggttttgatttgtaattcaccaatgactaaggatgttgaacatttctttaggtgcttcttggccatttgatattcctcagttgaaaattctttgtttagctctctaccccattttttaatagggttatttggttctctggagtctaacttcttgagttctttgtatatattggatattagccctctatcggatgtaggattggtaaagatcttttcccaatctgttggtattgttttgtccttttgtcagtgtcctttgccttacagaagctttgcaattttatgaggtcccacttgtcgattctttttttttttttttttttttttttttcgagacagggtttctctgtatagccctggctgtcctggagctcactttgtagaccaggctggcctcgaactcagaaatccgcctgcctctgcctcccgagtgctgggattaaaggcgtgcaccaccacacccggctccatttgtcgattcttgatcttacagcattagccattggtgttctattaaggaattttttcccctgtacccgtatgtttgaggctctttccaaattgctcctctataaagagagagagggggagagggagaaggagagagcaaggaaggaagaaaagagaagagacaagacaagaaggaaaaggagagaaaagaaaagagaaaaaagaaatgaaaagaagagaagagaaaagagaaaaaagaaaagaaagaccatgcacttgagggcactttgtttatgttgatagttcttggagaagctgagcgccgtctaagattcatgtaaaagcctcatgagaaatcagtgctttcacagacacatgtaaatagcaacaggcctgtgagcacagctacacaggggagctggctgcactccactagaattgccacgacgtacctatcacagtgacgtctacagtgagaaatgctgttctagactcattacatatgtaatcatttaaatgtaatcatttagagcctgggcagaaatctataacctatgaaaatgttcatttaaaatcaggtcaggcacaagtgtgcctggcttgttctcatcccagctgcctaggaatgtgggccaacctcagccacatagcaagagcctgtcgtacacatatgtacaggtgtagaggtatgtgtgcatgtatataggtactatataaaatacagagcccagagcctgtgatgcttcagatggcctcggacttctctggagacctggggaacgtgttaacaaagccttccctaaccccatggaagccgtgtgtccctgtgtgcattggtcctttggtgtttgcctccctctgtattgcagaagctctttgagcagggtctcagttcctccctgtggccccccagcccctgtgacagtgcagcgctggcacagacagtgcccacagctaccgaatgaatgagatctgaaattgaagcagatgactgttctggctgtttgtgtaatgaaataaccacgtttgtgtgtttctatgcatcacctttgtgtctgcatccttttatatttttgtctggagccccactgttgtatatcgacaaacctaacacacaccggaaccaggactctggatctcaggtgctgctcctttaaactggaatccgtgaggcagaggtgccgtctacaacccgttcctgacagcaggacttgcattctggcattctggatccacactgctgtcccttccaagtgttactgtctctcattcctctgtctctgagatcccttctgctctacctcaagagactaagagggtgaacacagtttgtacttttcatcttccttacagagaaggaaataataaattcatatttgttcccaagaaaaaaaaaaaaaa

SEQ ID NO: 82 - DNA, 2755 bp, Homo sapiens
  source 1..2755: mol_type = genomic DNA
ttaggacggggcgatggcggctgagaggagctgcgcgtgcgcgaacatgtaactggtgggatctgcggcggctcccagatgatggtcgtcctcctgggcgcgacgaccctagtgctcgtcgccgtgggcccatgggtgttgtccgcagccgcaggtggaaaaaatctaaaatctcctcaaaaagtagaggtcgacatcatagatgacaactttatcctgaggtggaacaggagcgatgagtctgtcgggaatgtgactttttcattcgattatcaaaaaactgggatggataattggataaaattgtctgggtgtcagaatattactagtaccaaatgcaacttttcttcactcaagctgaatgtttatgaagaaattaaattgcgtataagagcagaaaaagaaaacacttcttcatggtatgaggttgactcatttacaccatttcgcaaagctcagattggtcctccagaagtacatttagaagctgaagataaggcaatagtgatacacatctctcctggaacaaaagatagtgttatgtgggctttggatggtttaagctttacatatagcttacttatctggaaaaactcttcaggtgtagaagaaaggattgaaaatatttattccagacataaaatttataaactctcaccagagactacttattgtctaaaagttaaagcagcactacttacgtcatggaaaattggtgtctatagtccagtacattgtataaagaccacagttgaaaatgaactacctccaccagaaaatatagaagtcagtgtccaaaatcagaactatgttcttaaatgggattatacatatgcaaacatgacctttcaagttcagtggctccacgcctttttaaaaaggaatcctggaaaccatttgtataaatggaaacaaatacctgactgtgaaaatgtcaaaactacccagtgtgtctttcctcaaaacgttttccaaaaaggaatttaccttctccgcgtacaagcatctgatggaaataacacatctttttggtctgaagagataaagtttgatactgaaatacaagctttcctacttcctccagtctttaacattagatcccttagtgattcattccatatctatatcggtgctccaaaacagtctggaaacacgcctgtgatccaggattatccactgatttatgaaattattttttgggaaaacacttcaaatgctgagagaaaaattatcgagaaaaaaactgatgttacagttcctaatttgaaaccactgactgtatattgtgtgaaagccagagcacacaccatggatgaaaagctgaataaaagcagtgtttttagtgacgctgtatgtgagaaaacaaaaccaggaaatacctctaaaatttggcttatagttggaatttgtattgcattatttgctctcccgtttgtcatttatgctgcgaaagtcttcttgagatgcatcaattatgtcttctttccatcacttaaaccttcttccagtatagatgagtatttctctgaacagccattgaagaatcttctgctttcaacttctgaggaacaaatcgaaaaatgtttcataattgaaaatataagcacaattgctacagtagaagaaactaatcaaactgatgaagatcataaaaaatacagttcccaaactagccaagattcaggaaattattctaatgaagatgaaagcgaaagtaaaacaagtgaagaactacagcaggactttgtatgaccagaaatgaactgtgtcaagtataaggtttttcagcaggagttacactgggagcctgaggtcctcaccttcctctcagtaactacagagaggacgtttcctgtttagggaaagaaaaaacatcttcagatcataggtcctaaaaatacgggcaagctcttaactatttaaaaatgaaattacaggcccgggcacggtggctcacacctgtaatcccagcactttgggaggctgaggcaggcagatcatgaggtcaagagatcgagaccagcctggccaacgtggtgaaaccccatctctactaaaaatacaaaaattagccgggtagtaggtaggcgcgcgcctgttgtcttagctactcaggaggctgaggcaggagaatcgcttgaaaacaggaggtggaggttgcagtgagccgagatcacgccactgcactccagcctggtgacagcgtgagactctttaaaaaaagaaattaaaagagttgagacaaacgtttcctacattcttttccatgtgtaaaatcatgaaaaagcctgtcaccggacttgcattggatgagatgagtcagaccaaaacagtggccacccgtcttcctcctgtgagcctaagtgcagccgtgctagctgcgcaccgtggctaaggatgacgtctgtgttcctgtccatcactgatgctgctggctactgcatgtgccacacctgtctgttcgccattcctaacattctgtttcattcttcctcgggagatatttcaaacatttggtcttttcttttaacactgagggtaggcccttaggaaatttatttaggaaagtctgaacacgttatcacttggttttctggaaagtagcttaccctagaaaacagctgcaaatgccagaaagatgatccctaaaaatgttgagggacttctgttcattcatcccgagaacattggcttccacatcacagtatctacccttacatggtttaggattaaagccaggcaatcttttactatg

SEQ ID NO: 83 - DNA, 20 bp, Homo sapiens
  source 1..20: mol_type = genomic DNA
tcatagatgacaactttatc

SEQ ID NO: 84 - DNA, 21 bp, Homo sapiens
  source 1..21: mol_type = genomic DNA
tcatagatgacaactttatcc

SEQ ID NO: 85 - DNA, 22 bp, Homo sapiens
  source 1..22: mol_type = genomic DNA
tcatagatgacaactttatcct

SEQ ID NO: 86 - DNA, 23 bp, Homo sapiens
  source 1..23: mol_type = genomic DNA
tcatagatgacaactttatcctg

SEQ ID NO: 87 - DNA, 24 bp, Homo sapiens
  source 1..24: mol_type = genomic DNA
tcatagatgacaactttatcctga

SEQ ID NO: 88 - DNA, 25 bp, Homo sapiens
  source 1..25: mol_type = genomic DNA
tcatagatgacaactttatcctgag

SEQ ID NO: 89 - DNA, 20 bp, Homo sapiens
  source 1..20: mol_type = genomic DNA
tgttcattcatcccgagaac

SEQ ID NO: 90 - DNA, 21 bp, Homo sapiens
  source 1..21: mol_type = genomic DNA
tgttcattcatcccgagaaca

SEQ ID NO: 91 - DNA, 22 bp, Homo sapiens
  source 1..22: mol_type = genomic DNA
tgttcattcatcccgagaacat

SEQ ID NO: 92 - DNA, 23 bp, Homo sapiens
  source 1..23: mol_type = genomic DNA
tgttcattcatcccgagaacatt

SEQ ID NO: 93 - DNA, 24 bp, Homo sapiens
  source 1..24: mol_type = genomic DNA
tgttcattcatcccgagaacattg

SEQ ID NO: 94 - skipped

SEQ ID NO: 95 - DNA, 25 bp, Homo sapiens
  source 1..25: mol_type = genomic DNA
tgttcattcatcccgagaacattgg

SEQ ID NO: 96 - RNA, 20 nt, synthetic construct
  source 1..20: mol_type = other RNA
tcatagatgacaactttatc

SEQ ID NO: 97 - RNA, 20 nt, synthetic construct
  source 1..20: mol_type = other RNA
gataaagttgtcatctatga

SEQ ID NO: 98 - RNA, 21 nt, synthetic construct
  source 1..21: mol_type = other RNA
tcatagatgacaactttatcc

SEQ ID NO: 99 - RNA, 21 nt, synthetic construct
  source 1..21: mol_type = other RNA
ggataaagttgtcatctatga

SEQ ID NO: 100 - RNA, 22 nt, synthetic construct
  source 1..22: mol_type = other RNA
tcatagatgacaactttatcct

SEQ ID NO: 101 - RNA, 22 nt, synthetic construct
  source 1..22: mol_type = other RNA
aggataaagttgtcatctatga

SEQ ID NO: 102 - RNA, 23 nt, synthetic construct
  source 1..23: mol_type = other RNA
tcatagatgacaactttatcctg

SEQ ID NO: 103 - RNA, 23 nt, synthetic construct
  source 1..23: mol_type = other RNA
caggataaagttgtcatctatga

SEQ ID NO: 104 - RNA, 24 nt, synthetic construct
  source 1..24: mol_type = other RNA
tcatagatgacaactttatcctga

SEQ ID NO: 105 - RNA, 24 nt, synthetic construct
  source 1..24: mol_type = other RNA
tcaggataaagttgtcatctatga

SEQ ID NO: 106 - RNA, 25 nt, synthetic construct
  source 1..25: mol_type = other RNA
tcatagatgacaactttatcctgag

SEQ ID NO: 107 - RNA, 25 nt, synthetic construct
  source 1..25: mol_type = other RNA
ctcaggataaagttgtcatctatga

SEQ ID NO: 108 - RNA, 20 nt, synthetic construct
  source 1..20: mol_type = other RNA
tgttcattcatcccgagaac

SEQ ID NO: 109 - RNA, 20 nt, synthetic construct
  source 1..20: mol_type = other RNA
gttctcgggatgaatgaaca

SEQ ID NO: 110 - RNA, 21 nt, synthetic construct
  source 1..21: mol_type = other RNA
tgttcattcatcccgagaaca

SEQ ID NO: 111 - RNA, 21 nt, synthetic construct
  source 1..21: mol_type = other RNA
tgttctcgggatgaatgaaca

SEQ ID NO: 112 - RNA, 22 nt, synthetic construct
  source 1..22: mol_type = other RNA
tgttcattcatcccgagaacat

SEQ ID NO: 113 - RNA, 22 nt, synthetic construct
  source 1..22: mol_type = other RNA
atgttctcgggatgaatgaaca

SEQ ID NO: 114 - RNA, 23 nt, synthetic construct
  source 1..23: mol_type = other RNA
tgttcattcatcccgagaacatt

SEQ ID NO: 115 - RNA, 23 nt, synthetic construct
  source 1..23: mol_type = other RNA
aatgttctcgggatgaatgaaca

SEQ ID NO: 116 - RNA, 24 nt, synthetic construct
  source 1..24: mol_type = other RNA
tgttcattcatcccgagaacattg

SEQ ID NO: 117 - RNA, 24 nt, synthetic construct
  source 1..24: mol_type = other RNA
caatgttctcgggatgaatgaaca

SEQ ID NO: 118 - RNA, 25 nt, synthetic construct
  source 1..25: mol_type = other RNA
tgttcattcatcccgagaacattgg

SEQ ID NO: 119 - RNA, 25 nt, synthetic construct
  source 1..25: mol_type = other RNA
ccaatgttctcgggatgaatgaaca

SEQ ID NO: 120 - RNA, 21 nt, synthetic construct
  source 1..21: mol_type = other RNA
  misc_feature 21: note = 3' dTdT
tcatagatgacaactttatcc

SEQ ID NO: 121 - RNA, 21 nt, synthetic construct
  source 1..21: mol_type = other RNA
  misc_feature 21: note = 3' dTdT
ggataaagttgtcatctatga

SEQ ID NO: 122 - RNA, 21 nt, synthetic construct
  source 1..21: mol_type = other RNA
tgttcattcatcccgagaaca

SEQ ID NO: 123 - RNA, 21 nt, synthetic construct
  source 1..21: mol_type = other RNA
tgttctcgggatgaatgaaca

(19) United States
(12) Patent Application Publication
(10) Pub. No.: US 2026/0364166 A1
(43) Pub. Date: Nov. 19, 2026

(54) RNA-LOADED LIPID NANOPARTICLES

(71) Applicant: Massachusetts Institute of Technology, Cambridge, MA (US)

(72) Inventors: Darrell IRVINE, Cambridge, MA (US); Byungji KIM, Cambridge, MA (US)

(21) Appl. No.: 19/487,111

(22) PCT Filed: Apr. 5, 2024
(86) PCT No.: PCT/US2024/023210

§ 371 Date: Nov. 24, 2025

Related U.S. Application Data
(60) Provisional application No. 63/506,661, filed on Jun. 7, 2023.

(51) Int. Cl. A61K 39/215; A61K 31/713; A61K 39/00; A61K 39/21; A61P 37/04; C12N 15/113

(52) U.S. Cl. CPC A61K 39/215; A61K 31/713; A61K 39/21; A61P 37/04; C12N 15/1138; A61K 2039/51; A61K 2039/6018; C12N 2310/11; C12N 2310/14; C12N 2310/3231

(57) ABSTRACT

Compositions are provided that include a lipid nanoparticle (LNP); a self-replicating RNA encoding a gene of interest loaded within the LNP; and an inhibitory nucleic acid, such as a small interfering RNA (siRNA) targeting IFN-α/β receptor 1 (Ifhar1) gene loaded within the LNP, and use of the compositions for generating an immune response.

### FEDERAL FUNDING STATEMENT

This invention was made with government support under EB025854, CA265706, and AI144462 awarded by the National Institutes of Health. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Mar. 13, 2024 having the file name “23-0811-WO.xml” and is 122,127 bytes in size.

### BACKGROUND

RNA vaccines have emerged as a breakthrough technology in the COVID-19 pandemic. An advancement on this technology uses self-replicating RNA (repRNA) in lieu of mRNAs. Compared to the traditional mRNA, repRNA has the potential for much more prolonged expression, which could be effective for promoting bumoral immunity to vaccine immunogens. The repRNA is inherently based on the alphaviral RNA, in which the non-structural protein (nsP) segments of the back-bone enable replication of the gene of interest (FIG. 1a). The segment also allows the repRNA construct to be recognized as a viral infection via activation of toll-like receptor-7 (TLR7)-induced response. However, this innate immune recognition may hinder the response by inhibiting RNA replication and translation, or by overstimulating innate immunity (FIG. 1b).

### SUMMARY

In one aspect, the disclosure provides compositions, comprising:
(a) a lipid nanoparticle (LNP);
(b) a self-replicating RNA encoding a gene of interest loaded within the LNP; and
(c) an inhibitory nucleic acid targeting IFN-α/β receptor 1 (Ifnar1) gene loaded within the LNP. In one embodiment, the inhibitory nucleic acid comprises a short interfering RNA (siRNA). In another embodiment, the inhibitory nucleic acid targets a region of the Ifnar1 gene comprising the nucleotide sequence selected from SEQ ID NO:1-4, 83, and 89. In a further embodiment, the inhibitory nucleic acid targets a region of the Ifnar1 gene comprising the nucleotide sequence selected from SEQ ID NO:1-24 and 83-95.

In one embodiment, the inhibitory nucleic acid comprises a double stranded siRNA sequence comprising the nucleotide sequence of a pair of sequences selected from the following pairs:

(a) sense (5′→3′):
(SEQ ID NO: 25)
GUU GAU CCG UUU AUU CCA UU
and
anti-sense (5′→3′):
(SEQ ID NO: 26)
AAU GGA AUA AAC GGA UCA AC
(IFNAR1.1 mouse minimal sequence);
(b) sense (5′→3′):
(SEQ ID NO: 27)
GUA GAA GUA AAG CAC GCG CC
and
anti-sense (5′→3′):
(SEQ ID NO: 28)
GGC GCG UGC UUU ACU UCU AC
(IFNAR1.2 mouse minimal sequence);
(c) sense (5′→3′):
(SEQ ID NO: 29)
CAA AGC UCA GAU UGG UCC UC
and
anti-sense (5′→3′): anti-sense (5′→3′):
(SEQ ID NO: 30)
GAG GAC CAA UCU GAG CUU UG
(IFNAR1.1 human minimal sequence);
(d) sense (5′→3′):
(SEQ ID NO: 31)
GUG CUC CAA AAC AGU CUG GA
and
anti-sense (5′→3′): (5′→3′):
(SEQ ID NO: 32)
UCC AGA CUG UUU UGG AGC AC
(IFNAR1.2 human minimal sequence);
(e) sense (5′→3′):
(SEQ ID NO: 96)
UCA UAG AUG ACA ACU UUA UC
and
anti-sense (5′→3′):
(SEQ ID NO: 97)
GAU AAA GUU GUC AUC UAU GA
(IFNAR1.3 human minimal sequence);
and
(f) sense (5′→3′):
(SEQ ID NO: 108)
UGU UCA UUC AUC CCG AGA AC
and
anti-sense (5′→3′):
(SEQ ID NO: 109)
GUU CUC GGG AUG AAU GAA CA
(IFNAR1.4 human minimal sequence).

In one embodiment, the 5′ end of the sequences are phosphorylated. In another embodiment, the nucleic acids comprise a two nucleotide single stranded overhang at the 3′ terminus. In a further embodiment, the two nucleotide single stranded overhang at the 3′ terminus comprises dTdT. In various embodiments, the inhibitory nucleic acid comprises a double stranded siRNA sequence comprising the nucleotide sequence of a pair of sequences selected from the following pairs:

(a) sense (5′→3′):
(SEQ ID NO: 72)
GUU GAU CCG UUU AUU CCA UUC dTdT
and
anti-sense (5′→3′):
(SEQ ID NO: 73)
GAA UGG AAU AAA CGG AUC AAC dTdT
(IFNAR1.1 mouse 21 nt sequence with overhang);
(b) sense (5′→3′):
(SEQ ID NO: 74)
GUA GAA GUA AAG CAC GCG CCU dTdT
and
anti-sense (5′→3′):
(SEQ ID NO: 75)
AGG CGC GUG CUU UAC UUC UAC dTdT;
(IFNAR1.2 mouse 21 nt sequence with overhang);
(c) sense (5′→3′):
(SEQ ID NO: 76)
CAA AGC UCA GAU UGG UCC UCC dTdT
and
anti-sense (5′→3′):
(SEQ ID NO: 77)
GGA GGA CCA AUC UGA GCU UUG dTdT
(IFNAR1.1 human 21 nt sequence with overhang);
and
(d) sense (5′→3′):
(SEQ ID NO: 78)
CAA AGC UCA GAU UGG UCC UCC dTdT
and
anti-sense (5′→3′):
(SEQ ID NO: 79)
GGA GGA CCA AUC UGA GCU UUG dTdT
(IFNAR1.2 human 21 nt sequence with overhang).
In one embodiment, the 5′ end of the siRNA is
phosphorylated, and the 3′ end is hydroxylated.

In another embodiment, the siRNA comprises one or more locked nucleic acids (LNA). In a further embodiment, the gene of interest encodes an antigen, including but not limited to an immunogenic portion of a viral, bacterial, parasitic, protozoan, fungal, or tumor antigen. In some embodiments, the antigen comprises a human immunodeficiency virus (HIV) or a severe acute respiratory syndrome (SARS) antigen.

In one embodiment, wherein the LNP comprises an ionizable lipid, a helper lipid, cholesterol, and a polymer-conjugated lipid. In another embodiment, the helper lipid comprises phosphocholines, phosphoethanolamines, or combinations thereof. In a further embodiment, the polymer-conjugated lipid comprises polyethylene glycol or polysarcosine conjugated to phosphoethanolamines, 1,2-dimyristoyl-rac-glycero, or other amphiphilic molecules. In one embodiment, the amine-to-phosphate (N: P) ratio in the LNP loaded with RNA ranges from about 1:1 to about 20:1. In another embodiment, the lipid-to-RNA volume ratio in the LNP may range from about 1:1 to about 1:20. In a further embodiment, the LNP comprises
(a) N1,N3,N5-tris(3-(didodecylamino) propyl)benzene-1,3,5-tricarboxamide (TT3), (b) (6Z,9Z,28Z,31Z)-Heptatriaconta-6,9,28,31-tetraen-19-yl 4-(dimethylamino)butanoate (DLin-MC3-DMA),
(c) 1,2-dioleoyl-sn-glycero-3-phosphoethanolamine (DOPE),
(d) cholesterol, and
(e) 1,2-dimyristoyl-rac-glycero-3-methoxypolyethylene glycol-2000 (DMG-PEG2k);

In one embodiment, the disclosure provides vaccines comprising a composition of the disclosure where the gene of interest encodes an antigen. The disclosure also provides methods for generating an immune response against an antigen, comprising administering to a subject an amount effective to generate an immune response in the subject of the composition or vaccine of any embodiment herein where the gene of interest encodes an antigen. In another embodiment, the methods comprise of treating an infection or limiting development of an infection in a subject in need thereof comprising administering to the subject the composition or vaccine of any embodiment herein where the gene of interest encodes an antigen in an effective amount to induce an immune response against the antigen. In one embodiment, the antigen comprises an HIV antigen, and the subject is at risk of, or has, an HIV infection. In another embodiment, the antigen comprises a SARS-COV-2 antigen, and the subject is at risk of, or has, an SARS-COV-2 infection. The subject may be any mammalian subject, including but not limited to a human subject.

### DESCRIPTION OF THE FIGURES

FIG. 1. Self-replicating RNA (repRNA) and the type I interferon (IFN) pathway recognition of repRNA vaccines. (a) Structure of the native alphaviral RNA and the engineered repRNA, wherein the viral structural genes are replaced with any gene of interest; (b) schematic illustrating interferon-α (IFN-α) and interferon-β (IFN-β)-mediated triggering of dimerization between IFN-α/B receptor 1 (IFNAR1) and IFNAR2. Downstream JAK and STAT1 signaling activated by IRF9 induces Interferon-stimulated response element (ISRE) to transcribe interferon-stimulated genes (ISGs). ISGs inhibit replication inhibition and modulate immune response to viral infections. It may also suppress repRNA replication from lipid nanoparticle (LNP) vaccines (1). On the other hand, MYD88 triggering by endocytosed LNP vaccines may promote adjuvant responses, including early differentiation of follicular helper T (Tfh) cells (2).

FIG. 2. Characterization of LNP-repRNA vaccines synthesized to study the role of type I IFN in vaccine-elicited immune response. (a) Three LNP formulations were developed for comparative testing: “LNP-repRNA” refers to LNPs loaded with repRNA encoding for either the HIV immunogen or a reporter protein; “LNP-repRNA/siRNA” refers to LNPs that load a combination of the repRNA and siRNA against Ifnar1 (two candidate siRNA sequences are tested, labeled as siIFNAR1.1, and siIFNAR1.2, as well as a scramble of the siIFNAR1.2 sequence, labelled siScramble); and “LNP-repRNA+LNP-siRNA” refers to a cocktail mixture of LNPs loaded with only the repRNA and LNPs loaded with only the siIFNAR1.2; (b-c) Groups balb/c mice (n=3 animals/group averaged across a total of 6 legs/group) were injected i.m. in both the left and right gastrocnemius muscles with 1 μg replicon RNA in LNPs. Shown are representative photograph/false-color overlays (b), and luciferase reporter signals over time (c); dotted line indicates background signal of untreated mice, shown are means±standard deviation (n=6). (d-e) Cryogenic electron microscope (Cryo-EM) images of: (d) LNP-repAg, and (e) LNP-repAg/siIFNAR1. (f) Dynamic light scattering (DLS)-measured number-weighted size distribution of LNP-repAg (dotted black line) and LNP-repAg/siIFNAR1 (red solid line).

FIG. 3. Silencing Ifnar1 in C2C12 mouse myoblasts enhances expression of repRNA in vitro. (a-c) C2C12 mouse myoblasts were treated with PBS, LNPs loaded with repRNA encoding for GFP (repGFP), or LNPs that co-load repGFP and siRNA against Ifnar1 (repGFP/siIFNAR1). At 24 h post-transfection, cells were analyzed by flow cytometry for: (a) cell viability using zombie Aqua staining; (b) LNP uptake into cells using DiI as a lipophilic label on the LNPs; and (c) Protein expression by assessing the percentage of GFP+ population of live cells. (d-e) C2C12 mouse myoblasts were treated with PBS, LNPs loaded with repRNA encoding for GFP (repGFP), LNPs that co-load repGFP and siRNA against Ifhar1 (repGFP/siIFNAR1.1 and repGFP/siIFNAR1.2), LNPs that co-load repGFP and siRNA encoding scrambled of the sequence against Ifnar1 (repGFP/siScramble), or a mixture of LNPs loaded with only the repRNA and LNPs loaded with only the siIFNAR1.2. At 24 h post-transfection, cells were lysed and RNA was purified. Quantitative real-time polymerase chain reaction (qRT-PCR) was used to quantify relative RNA expression levels of: (d) Ifnar1; (e) the repRNA backbone encoding for the replicase; and (f) Gfp as the model repRNA payload. Samples are presented as means #standard deviation. Statistics represent one-way ANOVA and Tukey's HSD Test (ns=not significant; *, p<0.05; **, p<0.01; ***, p<0.001; ****, p<0.0001).

FIG. 4. In vivo knockdown of Ifnar1 increases expression level of repRNA when packaged and delivered with siIFNAR1 in the same LNP. (a-1) Groups of balb/C mice (n=5 animals/group) were immunized i.m. in each leg with 1 μg repRNA loaded in LNPs, and sacrificed at days 1, 3, 7, and 14 post-injection for gastrocnemius (muscle) and popliteal lymph node (pLN) harvesting. RNA was purified from the tissues for qRT-PCR-based quantification of the relative expression levels of: (a) Ifnar1 in the muscle; (b) the repRNA backbone encoding for the replicase in the muscle; (c) Gfp as the model repRNA payload in the muscle; (d) Ifnar1 in the pLN; (e) the repRNA backbone in the pLN; and (f) Gfp in the pLN. For expression levels of other key markers in the muscle, we evaluated: (g) Jak1; (h) Stat1; (i) Irf9; (1) Myd88; (k) Ifnα; (1) ifnβ. (m) Profile of cytokine production from the gastrocnemius muscles of mice at days 1, 3, and 7 post-vaccination with LNPs.

FIG. 5. Silencing Ifnar1 induces significantly more robust antibody production, antigen-specific B cell expansion, and antigen-specific T cell response. Groups of BALB/c mice (n=5 animals/group) were immunized i.m. in each leg with 1 μg repRNA. Shown are frequency of total GC B cells (a), follicular helper T cells (b), antigen-specific B cells (c), high-binding antigen-specific B cells (d). Histogram of antigen-specific B cells is shown in (e). Serum antibody responses were quantified by ELISA assay conducted on mouse sera collected from 0 to 112 days post-vaccination (f). **, p<0.01; ***, p<0.001; ****, p<0.0001 by two-way ANOVA with Tukey's post test. (g) Endpoint serum IgG titer at day 14 post-vaccination. Statistics represent two-way ANOVA and Tukey's HSD Test (*, p<0.05; **, p<0.01; ***, p<0.001; ****, p<0.0001). Percentage of antigen-specific IgG out of total IgG secreted by bone-marrow plasma cells (b) and antigen-specific T cell (splenocyte) activity (i-j) were quantified by ELISpot: (i) a representative image of the ELISpot wells for each group; and (j) counted number of spots per 10⁶ cells in response to overlapping peptides for the HIV immunogen. Each column graph shows means±s.e.m. Statistics represent one-way ANOVA and Tukey's HSD Test (ns=not significant; *, p<0.05; **, p<0.01; ***, p<0.001; ****, p<0.0001).

### DETAILED DESCRIPTION

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

All embodiments of any aspect of the disclosure can be used in combination, unless the context clearly dictates otherwise.

As used herein, “about” will mean up to plus or minus 5% of the particular value.

In one aspect, the disclosure provides compositions, comprising:
(a) a lipid nanoparticle (LNP);
(b) a self-replicating RNA (repRNA) encoding a gene of interest loaded within the LNP; and
(c) an inhibitory nucleic acid targeting IFN-α/β receptor 1 (Ifhar1) gene loaded within the LNP.

As shown in the examples that follow, the inventors have demonstrated that the compositions of the invention silence IFN-α/β receptor 1 (Ifhar1) to suppress type 1 interferon response upon administration of the compositions, permitting higher levels and more prolonged expression of repRNAs, which could be effective for promoting humoral immunity to vaccine immunogens.

Replicon RNA (repRNA) is a promising vaccine platform for safety and efficacy. RepRNA vaccine encodes not only antigen genes but also the genes necessary for RNA replication. Thus, repRNA is self-replicative and can play the role of an adjuvant by itself, which elicits robust immunity.

The mouse and human Ifnar1 gene sequences are provided in Table I below.

The inhibitory nucleic acid may be any inhibitory nucleic acid that can serve to inhibit Ifnar1 expression, including but not limited to antisense nucleic acids and a short interfering RNA (siRNA).

In some embodiments, the inhibitory nucleic acid targets a region of the Ifnar1 gene comprising the nucleotide sequence selected from SEQ ID NO:1-4.
gttgatcegt ttattccatt (SEQ ID NO:1) (IFNAR1.1 minimal sequence, mouse);
gtagaagtaa agcacgcgcc (SEQ ID NO:2) (IFNAR1.2 minimal sequence, mouse);
gtgctccaaa acagtctgga (SEQ ID NO:3) (IFNAR1.1 minimal sequence, human);
tcatagatga caactttate (SEQ ID NO:4) (IFNAR1.2 minimal sequence, human);
tcatagatga caactttatc (SEQ ID NO:83) (IFNAR1.3 minimal sequence, human); and
tgttcattca tecegagaac (SEQ ID NO:89 (IFNAR1.4 minimal sequence, human).

The nucleic acid sequence of SEQ ID NO: 1 is in exon 3 in the mouse Ifnar1 gene. The nucleic acid sequence of SEQ ID NO:2 is in exon 11 or 12 in the mouse Ifhar1 gene. The nucleic acid sequence of SEQ ID NO:3 is in exon3 or 4 in the human Ifhar1 gene. The nucleic acid sequence of SEQ ID NO:4 is in exon 8 in the human Ifnar1 gene. The nucleic acid sequence of SEQ ID NO:83 is in exon 2 in the human Ifnar1 gene. The nucleic acid sequence of SEQ ID NO:89 is in exon 11 in the human Ifnar1 gene.

In other embodiments, the inhibitory nucleic acid targets a region of the Ifhar1 gene comprising the nucleotide sequence selected from SEQ ID NO: 1-24 and 83-95.

IFNAR1. 1 (mouse) Exon 3
RefSeq: NM_010508.2

| SEQ ID NO | Sequence |
|---|---|
| 1 | gttgatccgt ttattccatt |
| 5 | gttgatccgt ttattccatt c |
| 6 | gttgatccgt ttattccatt ct |
| 7 | gttgatccgt ttattccatt cta |
| 8 | gttgatccgt ttattccatt ctac |
| 9 | gttgatccgt ttattccatt ctaca |

IFNAR1.2 (mouse) Exon 11
RefSeq: NM_010508.2

| SEQ ID NO | Sequence |
|---|---|
| 2 | gtagaagtaa agcacgcgcc |
| 10 | gtagaagtaa agcacgcgcc t |
| 11 | gtagaagtaa agcacgcgcc tg |
| 12 | gtagaagtaa agcacgcgcc tga |
| 13 | gtagaagtaa agcacgcgcc tgag |
| 14 | gtagaagtaa agcacgcgcc tgagg |

IFNAR1.1 (human) Exon 3 or 4
RefSeq: NP_000620.2; XP_005261021.1;
XP_011527854.1-

| SEQ ID NO | Sequence |
|---|---|
| 3 | caaagctcag attggtcctc |
| 15 | caaagctcag attggtcctc c |
| 16 | caaagctcag attggtcctc ca |
| 17 | caaagctcag attggtcctc cag |
| 18 | caaagctcag attggtcctc caga |
| 19 | caaagctcag attggtcctc cagaa |

IFNAR1.2 (human) Exon 8
RefSeq: NP_000620.2; XP_005261021.1

| SEQ ID NO | Sequence |
|---|---|
| 4 | gtgctccaaa acagtctgga |
| 20 | gtgctccaaa acagtctgga a |
| 21 | gtgctccaaa acagtctgga aa |
| 22 | gtgctccaaa acagtctgga aac |
| 23 | gtgctccaaa acagtctgga aaca |
| 24 | gtgctccaaa acagtctgga aacac |

IFNAR1.3 (human) Exon 2
RefSeq: NM_000629.2, XM_005260964.2,
XM_011529552.1

| SEQ ID NO | Sequence |
|---|---|
| 83 | tcatagatga caactttatc |
| 84 | tcatagatga caactttatc c |
| 85 | tcatagatga caactttatc ct |
| 86 | tcatagatga caactttatc ctg |
| 87 | tcatagatga caactttatc ctga |
| 88 | tcatagatga caactttatc ctgag |

IFNAR1.4 (human) Exon 11
RefSeq: NM_000629.2; XM_005260964.2

| SEQ ID NO | Sequence |
|---|---|
| 89 | tgttcattca tcccgagaac |
| 90 | tgttcattca tcccgagaac a |
| 91 | tgttcattca tcccgagaac at |
| 92 | tgttcattca tcccgagaac att |
| 93 | tgttcattca tcccgagaac attg |
| 95 | tgttcattca tcccgagaac attgg |

In one specific embodiment, the inhibitory nucleic acid comprises a short interfering RNA (siRNA).

siRNA is a class of double-stranded RNA at first non-coding RNA molecules, typically 20-25 base pairs in length, similar to miRNA, and operating within the RNA interference (RNAi) pathway. It interferes with the expression of specific genes with complementary nucleotide sequences by degrading mRNA after transcription, preventing translation. siRNAs have a well-defined structure that is a short (usually 20 to 25-bp) double-stranded RNA (dsRNA), and may have phosphorylated 5′ ends and hydroxylated 3′ ends with two overhanging nucleotides.

In some embodiments, the inhibitory nucleic acid comprises a double stranded siRNA sequence comprising the nucleotide sequence of a pair of sequences selected from the following pairs;

(a) sense (5′→3′):
(SEQ ID NO: 25)
GUU GAU CCG UUU AUU CCA UU
and
anti-sense (5′→3′):
(SEQ ID NO: 26)
AAU GGA AUA AAC GGA UCA AC
(IFNAR1.1 mouse minimal sequence);
(b) sense (5′→3′):
(SEQ ID NO: 27)
GUA GAA GUA AAG CAC GCG CC
and
anti-sense (5′→3′):
(SEQ ID NO: 28)
GGC GCG UGC UUU ACU UCU AC
(IFNAR1.2 mouse minimal sequence);
(c) sense (5′→3′):
(SEQ ID NO: 29)
CAA AGC UCA GAU UGG UCC UC
and
anti-sense (5′→3′): anti-sense (5′→3′):
(SEQ ID NO: 30)
GAG GAC CAA UCU GAG CUU UG
(IFNAR1.1 human minimal sequence);
(d) sense (5′→3′):
(SEQ ID NO: 31)
GUG CUC CAA AAC AGU CUG GA
and
anti-sense (5′→3′): (5′→3′):
(SEQ ID NO: 32)
UCC AGA CUG UUU UGG AGC AC
(IFNAR1.2 human minimal sequence);
(e) sense (5′→3′):
(SEQ ID NO: 96)
UCA UAG AUG ACA ACU UUA UC
and
anti-sense (5′→3′):
(SEQ ID NO: 97)
GAU AAA GUU GUC AUC UAU GA
(IFNAR1.3 human minimal sequence);
and
(f) sense (5′→3′):
(SEQ ID NO: 108)
UGU UCA UUC AUC CCG AGA AC
and
anti-sense (5′→3′):
(SEQ ID NO: 109)
GUU CUC GGG AUG AAU GAA CA
(IFNAR1.4 human minimal sequence).

In various other embodiments, the inhibitory nucleic acid comprises a double stranded siRNA sequence comprising the nucleotide sequence of a pair of sequences selected from the following pairs:

(a) sense (5′→3′):
(SEQ ID NO: 25)
GUU GAU CCG UUU AUU CCA UU
and
anti-sense (5′→3′):
(SEQ ID NO: 26)
AAU GGA AUA AAC GGA UCA AC
(IFNAR1.1 mouse minimal sequence);
(b) sense (5′→3′):
(SEQ ID NO: 27)
GUA GAA GUA AAG CAC GCG CC
and
anti-sense (5′→3′):
(SEQ ID NO: 28)
GGC GCG UGC UUU ACU UCU AC
(IFNAR1.2 mouse minimal sequence);
(c) sense (5′→3′):
(SEQ ID NO: 29)
CAA AGC UCA GAU UGG UCC UC
and
anti-sense (5′→3′): anti-sense (5′→3′):
(SEQ ID NO: 30)
GAG GAC CAA UCU GAG CUU UG
(IFNAR1.1 human minimal sequence);
(d) sense (5′→3′):
(SEQ ID NO: 31)
GUG CUC CAA AAC AGU CUG GA
and
anti-sense (5′→3′): (5′→3′):
(SEQ ID NO: 32)
UCC AGA CUG UUU UGG AGC AC
(IFNAR1.2 human minimal sequence);
(e) sense (5′→3′):
(SEQ ID NO: 80)
GUU GAU CCG UUU AUU CCA UUC
and
anti-sense (5′→3′):
(SEQ ID NO: 33)
GAA UGG AAU AAA CGG AUC AAC
(IFNAR1.1 mouse 21 nt sequence);
(f) sense (5′→3′):
(SEQ ID NO: 34)
GUU GAU CCG UUU AUU CCA UUC U
and
anti-sense (5′→3′):
(SEQ ID NO: 35)
AGA AUG GAA UAA ACG GAU CAA C
(IFNAR1.1 mouse 22 nt sequence);
(g) sense (5′→3′):
(SEQ ID NO: 36)
GUU GAU CCG UUU AUU CCA UUC UA
and
anti-sense (5′→3′):
(SEQ ID NO: 37)
UAG AAU GGA AUA AAC GGA UCA AC
(IFNAR1.1 mouse 23 nt sequence);
(h) sense (5′→3′):
(SEQ ID NO: 38)
GUU GAU CCG UUU AUU CCA UUC UAC
and
anti-sense (5′→3′):
(SEQ ID NO: 39)
GUA GAA UGG AAU AAA CGG AUC AAC
(IFNAR1.1 mouse 24 nt sequence);
(i) sense (5′→3′):
(SEQ ID NO: 40)
GUU GAU CCG UUU AUU CCA UUC UAC A
and
anti-sense (5′→3′):
(SEQ ID NO: 41)
UGU AGA AUG GAA UAA ACG GAU CAA C
(IFNAR1.1 mouse 25 nt sequence);
(j) sense (5′→3′):
(SEQ ID NO: 42)
GUA GAA GUA AAG CAC GCG CCU
and
anti-sense (5′→3′):
(SEQ ID NO: 43)
AGG CGC GUG CUU UAC UUC UAC;
(IFNAR1.2 mouse 21 nt sequence);
(k) sense (5′→3′):
(SEQ ID NO: 44)
GUA GAA GUA AAG CAC GCG CCU G
and
anti-sense (5′→3′):
(SEQ ID NO: 45)
CAG GCG CGU GCU UUA CUU CUA C
(IFNAR1.2 mouse 22 nt sequence);
(l) sense (5′→3′):
(SEQ ID NO: 46)
GUA GAA GUA AAG CAC GCG CCU GA
and
anti-sense (5′→3′):
(SEQ ID NO: 47)
UCA GGC GCG UGC UUU ACU UCU AC
(IFNAR1.2 mouse 23 nt sequence);
(m) sense (5′→3′):
(SEQ ID NO: 48)
GUA GAA GUA AAG CAC GCG CCU GAG
and
anti-sense (5′→3′):
(SEQ ID NO: 49)
CUC AGG CGC GUG CUU UAC UUC UAC
(IFNAR1.2 mouse 24 nt sequence);
(n) sense (5′→3′):
(SEQ ID NO: 50)
GUA GAA GUA AAG CAC GCG CCU GAG G
and
anti-sense (5′→3′):
(SEQ ID NO: 51)
CCU CAG GCG CGU GCU UUA CUU CUA C
(IFNAR1.2 mouse 25 nt sequence);
(o) sense (5′→3′):
(SEQ ID NO: 52)
CAA AGC UCA GAU UGG UCC UCC
and
anti-sense (5′→3′):
(SEQ ID NO: 53)
GGA GGA CCA AUC UGA GCU UUG
(IFNAR1.1 human 21 nt sequence);
(p) sense (5′→3′):
(SEQ ID NO: 54)
CAA AGC UCA GAU UGG UCC UCC A
and
anti-sense (5′→3′):
(SEQ ID NO: 55)
UGG AGG ACC AAU CUG AGC UUU G
(IFNAR1.1 human 22 nt sequence);
(q) sense (5′→3′):
(SEQ ID NO: 56)
CAA AGC UCA GAU UGG UCC UCC AG
and
anti-sense (5′→3′):
(SEQ ID NO: 57)
CUG GAG GAC CAA UCU GAG CUU UG
(IFNAR1.1 human 23 nt sequence);
(r) sense (5′→3′):
(SEQ ID NO: 58)
CAA AGC UCA GAU UGG UCC UCC AGA
and
anti-sense (5′→3′):
(SEQ ID NO: 59)
UCU GGA GGA CCA AUC UGA GCU UUG
(IFNAR1.1 human 24 nt sequence);
(s) sense (5′→3′):
(SEQ ID NO: 60)
CAA AGC UCA GAU UGG UCC UCC AGA A
and
anti-sense (5′→3′):
(SEQ ID NO: 61)
UUC UGG AGG ACC AAU CUG AGC UUU G
(IFNAR1.1 human 25 nt sequence);
(t) sense (5′→3′):
(SEQ ID NO: 62)
GUG CUC CAA AAC AGU CUG GAA
and
anti-sense (5′→3′):
(SEQ ID NO: 63)
UUC CAG ACU GUU UUG GAG CAC
(IFNAR1.2 human 21 nt sequence);
(u) sense (5′→3′):
SEQ ID NO: 64)
GUG CUC CAA AAC AGU CUG GAA A
and
anti-sense (5′→3′):
(SEQ ID NO: 65)
UUU CCA GAC UGU UUU GGA GCA C
(IFNAR1.2 human 22 nt sequence);
(v) sense (5′→3′):
(SEQ ID NO: 66)
GUG CUC CAA AAC AGU CUG GAA AC
and
anti-sense (5′→3′):
(SEQ ID NO: 67)
GUU UCC AGA CUG UUU UGG AGC AC
(IFNAR1.2 human 23 nt sequence);
(w) sense (5′→3′):
SEQ ID NO: 68)
GUG CUC CAA AAC AGU CUG GAA ACA
and
anti-sense (5′→3′):
(SEQ ID NO: 69)
UGU UUC CAG ACU GUU UUG GAG CAC
(IFNAR1.2 human 24 nt sequence);
(x) sense (5′→3′):
(SEQ ID NO: 70)
GUG CUC CAA AAC AGU CUG GAA ACA C
and
anti-sense (5′→3′):
(SEQ ID NO: 71)
GUG UUU CCA GAC UGU UUU GGA GCA C
(IFNAR1.2 human 25 nt sequence);
(y) sense (5′→3′):
(SEQ ID NO: 96)
UCA UAG AUG ACA ACU UUA UC
and
anti-sense (5′→3′):
(SEQ ID NO: 97)
GAU AAA GUU GUC AUC UAU GA
(IFNAR1.3 human minimal sequence);
(z) sense (5′→3′):
(SEQ ID NO: 98)
UCA UAG AUG ACA ACU UUA UCC
and
anti-sense (5′→3′):
(SEQ ID NO: 99)
GGA UAA AGU UGU CAU CUA UGA
(IFNAR1.3 human 21 nt sequence);
(aa) sense (5′→3′):
(SEQ ID NO: 100)
UCA UAG AUG ACA ACU UUA UCC U
and
anti-sense (5′→3′):
(SEQ ID NO: 101)
AGG AUA AAG UUG UCA UCU AUG A
(IFNAR1.3 human 22 nt sequence);
(bb) sense (5′→3′):
(SEQ ID NO: 102)
UCA UAG AUG ACA ACU UUA UCC UG
and
anti-sense (5′→3′):
(SEQ ID NO: 103)
CAG GAU AAA GUU GUC AUC UAU GA
(IFNAR1.3 human 23 nt sequence);
(cc) sense (5′→3′):
(SEQ ID NO: 104)
UCA UAG AUG ACA ACU UUA UCC UGA
and
anti-sense (5′→3′):
(SEQ ID NO: 105)
UCA GGA UAA AGU UGU CAU CUA UGA
(IFNAR1.3 human 24 nt sequence);
(dd) sense (5′→3′):
(SEQ ID NO: 106)
UCA UAG AUG ACA ACU UUA UCC UGA G
and
anti-sense (5′→3′):
(SEQ ID NO: 107)
CUC AGG AUA AAG UUG UCA UCU AUG A
(IFNAR1.3 human 25 nt sequence);
(ee) sense (5′→3′):
(SEQ ID NO: 108)
UGU UCA UUC AUC CCG AGA AC
and
anti-sense (5′→3′):
(SEQ ID NO: 109)
GUU CUC GGG AUG AAU GAA CA
(IFNAR1.4 human minimal sequence);
(ff) sense (5′→3′):
(SEQ ID NO: 110)
UGU UCA UUC AUC CCG AGA ACA
and
anti-sense (5′→3′):
(SEQ ID NO: 111)
UGU UCU CGG GAU GAA UGA ACA
(IFNAR1.4 human 21 nt sequence);
(gg) sense (5′→3′):
(SEQ ID NO: 112)
UGU UCA UUC AUC CCG AGA ACA U
and
anti-sense (5′→3′):
(SEQ ID NO: 113)
AUG UUC UCG GGA UGA AUG AAC A
(IFNAR1.4 human 22 nt sequence);
(hh) sense (5′→3′):
(SEQ ID NO: 114)
UGU UCA UUC AUC CCG AGA ACA UU
and
anti-sense (5′→3′):
(SEQ ID NO: 115)
AAU GUU CUC GGG AUG AAU GAA CA
(IFNAR1.4 human 23 nt sequence);
(ii) sense (5′→3′):
(SEQ ID NO: 116)
UGU UCA UUC AUC CCG AGA ACA UUG
and
anti-sense (5′→3′):
(SEQ ID NO: 117)
CAA UGU UCU CGG GAU GAA UGA ACA
(IFNAR1.4 human 24 nt sequence);
and
(jj) sense (5′→3′):
(SEQ ID NO: 118)
UGU UCA UUC AUC CCG AGA ACA UUG G
and
anti-sense (5′→3′):
(SEQ ID NO: 119)
CCA AUG UUC UCG GGA UGA AUG AAC A
(IFNAR1.4 human 25 nt sequence).

In one embodiment, the 5′ end of the sequences are phosphorylated. In another embodiment, the nucleic acids comprise a two nucleotide single stranded overhang at the 3″ terminus. In one such embodiment, the two nucleotide single stranded overhang at the 3′ terminus comprises dTdT. In other embodiments, the two nucleotide single stranded overhang at the 3′ terminus may comprise UU, or may be complementary to the target mRNA.

In further embodiments, the inhibitory nucleic acid comprises a double stranded siRNA sequence comprising the nucleotide sequence of a pair of sequences selected from the following pairs:

(a) sense (5′→3′):
(SEQ ID NO: 72)
GUU GAU CCG UUU AUU CCA UUC dTdT
and
anti-sense (5′→3′):
(SEQ ID NO: 73)
GAA UGG AAU AAA CGG AUC AAC dTdT
(IFNAR1.1 mouse 21 nt sequence with overhang);
(b) sense (5′→3′):
(SEQ ID NO: 74)
GUA GAA GUA AAG CAC GCG CCU dTdT
and
anti-sense (5′→3′):
(SEQ ID NO: 75)
AGG CGC GUG CUU UAC UUC UAC dTdT;
(IFNAR1.2 mouse 21 nt sequence with overhang);
(c) sense (5′→3′):
(SEQ ID NO: 76)
CAA AGC UCA GAU UGG UCC UCC dTdT
and
anti-sense (5′→3′):
(SEQ ID NO: 77)
GGA GGA CCA AUC UGA GCU UUG dTdT
(IFNAR1.1 human 21 nt sequence with overhang);
(d) sense (5′→3′):
(SEQ ID NO: 78)
CAA AGC UCA GAU UGG UCC UCC dTdT
and
anti-sense (5′→3′):
(SEQ ID NO: 79)
GGA GGA CCA AUC UGA GCU UUG dTdT
(IFNAR1.2 human 21 nt sequence with overhang);
(e) sense (5′→3′):
(SEQ ID NO: 120)
UCA UAG AUG ACA ACU UUA UCC dTdT
and
anti-sense (5′→3′):
(SEQ ID NO: 121)
GGA UAA AGU UGU CAU CUA UGA dTdT
(IFNAR1.3 human 21 nt sequence with overhang);
and
(f) sense (5′→3′):
(SEQ ID NO: 122)
UGU UCA UUC AUC CCG AGA ACA
and
anti-sense (5′→3′):
(SEQ ID NO: 123)
UGU UCU CGG GAU GAA UGA ACA
(IFNAR1.4 human 21 nt sequence with overhang).

In some embodiments, the 5′ end of the siRNA strands may be phosphorylated. In other embodiments, the 5′ end of the siRNA strands are not phosphorylated. In another embodiment, the 3′ end of the siRNA strands are hydroxylated; in other embodiments they are not hydroxylated. In one embodiment, the 5′ end of the siRNA strands are phosphorylated and the 3′ end of the siRNA strands are hydroxylated.

The siRNAs may comprise chemically modified residues to enhance stability or other beneficial characteristics. Modifications may include, for example, end modifications, e.g., 5′-end modifications (phosphorylation, conjugation, inverted linkages) or 3′-end modifications (conjugation, DNA nucleotides, inverted linkages, etc.); sugar modifications (e.g., at the 2′-position or 4′-position) or replacement of the sugar; and/or backbone modifications, including modification or replacement of the phosphodiester linkages. In one embodiment, the siRNA may be modified to comprise one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation.

The repRNA may encode any gene of interest, including but not limited to a gene encoding a therapeutic protein, antigen, immunogen, reporter protein, cytokine, etc. In one embodiment, the gene of interest encodes an antigen. The antigen may be any antigen suitable for an intended purpose. In some embodiments, the antigen comprises an immunogenic portion of a viral, bacterial, parasitic, protozoan, fungal, or tumor antigen.

Exemplary viruses comprising suitable antigens include, but are not limited to, e.g., respiratory syncytial virus (RSV), hepatitis B virus (HBV), hepatitis C virus (HCV), Dengue virus, herpes simplex virus (HSV; e.g., HSV-I, HSV-II), molluscum contagiosum virus, vaccinia virus, variola virus, lentivirus, human immunodeficiency virus (HIV), human papilloma virus (HPV), cytomegalovirus (CMV), varicella zoster virus (VZV), rhinovirus, enterovirus, adenovirus, coronavirus (e.g., SARS), influenza virus (flu), para-influenza virus, mumps virus, measles virus, papovavirus, hepadnavirus, flavivirus, retrovirus, arenavirus (e.g., Lymphocytic Choriomeningitis Virus, Junin virus, Machupo virus, Guanarito virus, or Lassa virus), norovirus, yellow fever virus, rabies virus, Filovirus (e.g., Ebola virus or marbug virus), hepatitis C virus, hepatitis B virus, hepatitis A virus, Morbilliviruses (e.g., measles virus), Rubulaviruses (e.g., mumps virus), Rubiviruses (e.g., rubella virus), bovine viral diarrhea virus. For example, the antigen can be CMV glycoprotein gH, or gL; Parvovirus; HIV glycoprotein gp 120 or gp 140, HIV p55 gag, pol; or RSV-F antigen. In some embodiments, the antigen is a viral antigen. In some embodiments, the viral antigen is an HIV antigen comprising gp120 or gp140. In some engineered HIV antigen is an engineered variant of gp120 (engineered Outer Domain, eOD). In some embodiments, the engineered HIV antigen is an engineered variant of gp140 (SOSIP). Further description SOSIP is provided by WO201605704A3, US20160185825A1, Georgiev et al., (2015) J Virol 89 (10): 5318-5329, all of which are incorporated herein by reference in their entirety.

In some embodiments, the antigen is from a parasite. In some embodiments, the antigen is derived from a species from within the Plasmodium genus, such as P. falciparum. P. vivax, P. malariae or P. ovale. Thus the immunogenic composition may be used for preparation of a vaccine for immunizing against malaria.

In some embodiments, the antigen is from a bacterial pathogen. Exemplary bacterial pathogens include, e.g., Neisseria spp, including N. gonorrhea and N. meningitides; Streptococcus spp, including S. pneumoniae, S. pyogenes, S. agalactiae, S. mutans; Haemophilus spp, including H. influenzae type B, non typeable H. influenzae, H. ducreyi; Moraxella spp, including M. catarrhalis, also known as Branhamella catarrhalis; Bordetella spp, including B. pertussis, B. parapertussis and B. bronchiseptica; Mycobacterium spp., including M. tuberculosis, M. bovis, M. leprae, M. avium, M. paratuberculosis, M. smegmatis; Legionella spp, including L. pneumophila; Escherichia spp, including enterotoxic E. coli, enterohemorragic E. coli, enteropathogenic E. coli; Vibrio spp, including V. cholera, Shigella spp, including S. sonnei, S. dysenteriae, S. flexnerii; Yersinia spp, including Y. enterocolitica, Y. pestis, Y. pseudotuberculosis, Campylobacter spp, including C. jejuni and C. coli; Salmonella spp, including S. typhi, S. paratyphi, S. choleraesuis, S. enteritidis; Listeria spp., including L. monocytogenes; Helicobacter spp, including H. pylori; Pseudomonas spp, including P. aeruginosa, Staphylococcus spp., including S. aureus, S. epidermidis; Enterococcus spp., including E. faecalis, E. faecium; Clostridium spp., including C. tetani, C. botulinum, C. difficile; Bacillus spp., including B. anthracis; Corynebacterium spp., including C. diphtheriae; Borrelia spp., including B. burgdorferi, B. garinii, B. afzelii, B. andersonii, B. hermsii; Ehrlichia spp., including E. equi and the agent of the Human Granulocytic Ehrlichiosis; Rickettsia spp, including R. rickettsii; Chlamydia spp., including C. trachomatis, C. neumoniae, C. psittaci; Leptospira spp., including L. interrogans; Treponema spp., including T. pallidum, T. denticola, T. hyodysenteriae

In certain embodiments, the antigen is from a fungal pathogen. Exemplary fungal pathogens include, e.g., Aspergillus fumigatus, A. flavus, A. niger, A. terreus, A. nidulans, Coccidioides immitis, Coccidioides posadasii, Cryptococcus neoformans, Histoplasma capsulatum, Candida albicans, and Pneumocystis jirovecii.

In certain embodiments, the antigen is from a protozoan pathogen. Exemplary protozoan pathogens include, e.g., Toxoplasma gondii and Strongyloides stercoralis.

In certain embodiments, the antigen is from a multicellular parasitic pathogen. Exemplary multicellular parasitic pathogens include, e.g., trematodes (flukes), cestodes (tapeworms), nematodes (roundworms), and arthropods.

In some embodiments, the antigen is derived from a tumor antigen selected from: (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors; (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT; (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), mammaglobin, alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer); (d) shared antigens, for example, melanoma-melanocyte differentiation antigens such as MART-1/Melan A, gp100, MCIR, melanocyte-stimulating hormone receptor, tyrosinase, tyrosinase related protein-1/TRP1 and tyrosinase related protein-2/TRP2 (associated with, e.g., melanoma); (e) prostate associated antigens such as PAP, PSA, PSMA, PSH-P1, PSM-P1, PSM-P2, associated with e.g., prostate cancer; (f) immunoglobulin idiotypes (associated with myeloma and B cell lymphomas, for example). In certain embodiments, tumor immunogens include, but are not limited to, p15, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29\BCAA), CA 195, CA 242, CA-50, CAM43, CD68\KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, TPS, and the like.

In some embodiments, the antigen comprises a human immunodeficiency virus (HIV) or a severe acute respiratory syndrome (SARS) antigen.

The compositions may comprise any lipid nanoparticle (LNP). In one embodiment, the LNP comprises an ionizable lipid, a helper lipid, cholesterol, and a polymer-conjugated lipid. In this embodiment, helper lipid may comprise, for example phosphocholines, phosphoethanolamines, or combinations thereof. In another embodiment, the polymer-conjugated lipid may comprise, for example, polyethylene glycol or polysarcosine conjugated to phosphoethanolamines, 1,2-dimyristoyl-rac-glycero, or other amphiphilic or lipid-like molecules.

In some embodiments, the amine-to-phosphate (N: P) ratio in the LNP loaded with RNA may range from about 1:1 to about 20:1. In other embodiments, the lipid-to-RNA volume ratio in the LNP may range from about 1:1 to about 1:20 (i.e., the ratio between the volume of lipids (all components mixed at predetermined molar ratio) in ethanol, and the volume of the RNA (repRNA+siRNA) in water or acidic buffer).

In another embodiment, the LNP comprises
(a) N1,N3,N5-tris(3-(didodecylamino) propyl)benzene-1,3,5-tricarboxamide (TT3), (b) (6Z,9Z,28Z,31Z)-Heptatriaconta-6,9,28,31-tetraen-19-yl 4-(dimethylamino)butanoate (DLin-MC3-DMA),
(c) 1,2-dioleoyl-sn-glycero-3-phosphoethanolamine (DOPE),
(d) cholesterol, and
(e) 1,2-dimyristoyl-rac-glycero-3-methoxypolyethylene glycol-2000 (DMG-PEG2k);

In one exemplary embodiment, the molar ratio of LNP components is about 10 (TT3):25 (DLin-MC3-DMA):20 (DOPE):40 (cholesterol):5 (DMG-PEG2k).

The compositions may comprise any other components as appropriate for an intended purpose. In one embodiment, the composition may further comprise an adjuvant.

As used herein, the term “adjuvant” refers to any substance that acts to augment and/or direct antigen-specific immune responses when used in combination with specific antigens. When combined with a vaccine antigen, adjuvant increases the immune response to the vaccine antigen as compared to the response induced by the vaccine antigen alone. Adjuvants help drive immunological mechanisms and shape the output immune response to vaccine antigens. Exemplary antigens that might be used with the compositions of the disclosure may comprise aluminum salts (“alum”; aluminum hydroxide, aluminum phosphate), “Adjuvant System 04” (AS04), trehalose-6,6′-dimycolate (TDM), muramyl dipeptide (MDP), pluronic block copolymers, alum solution, aluminum hydroxide, ADJUMER® (polyphosphazene); aluminum phosphate gel; glucans from algae; algammulin; aluminum hydroxide gel (alum); highly protein-adsorbing aluminum hydroxide gel; low viscosity aluminum hydroxide gel; AF or SPT (emulsion of squalane (5%), Tween 80 (0.2%), Pluronic L121 (1.25%), phosphate-buffered saline, pH 7.4); AVRIDINE™ (propanediamine); BAY R1005™ ((N-(2-deoxy-2-L-leucylamino-b-D-glucopyranosyl)-N-octadecyl-dodecanoyl-amide hydroacetate); CALCITRIOL™ (1-alpha,2S-dihydroxy-vitamin D3); calcium phosphate gel; CAPIM (calcium phosphate nanoparticles); cholera holotoxin, cholera-toxin-A1-protein-A-D-fragment fusion protein, sub-unit B of the cholera toxin; CRL 1005 (block copolymer P1205); cytokine-containing liposomes; DDA (dimethyldioctadecylammonium bromide); DHEA (dehydroepiandrosterone); DMPC (dimyristoylphosphatidylcholine); DMPG (dimyristoylphosphatidylglycerol); DOC/alum complex (deoxycholic acid sodium salt); Freund's complete adjuvant; Freund's incomplete adjuvant; gamma inulin; Gerbu adjuvant (mixture of: i)N-acetylglucosaminyl-(P1-4)-N-acetylmuramyl-L-alanyl-D-glutamine (GMDP), ii) dimethyldioctadecylammonium chloride (DDA), iii) zinc-L-proline salt complex (ZnPro-8); GM-CSF); GMDP (N-acetylglucosaminyl-(b1-4)-N-acetylmuramyl-L-alanyl-D-isoglutamine); imiquimod (1-(2-methylpropyl)-1H-imidazol-4,5-c) quinoline-4-amine); ImmTher™ (N-acetylglucosaminyl-N-acetylmuramyl-L-Ala-D-isoGlu-L-Ala-glycerol dipalmitate); DRVs (immunoliposomes prepared from dehydration-rehydration vesicles); interferon-gamma; interleukin-1beta; interleukin-2; interleukin-7; interleukin-12; ISCOMS™; ISCOPREP 7.0.3.™; liposomes; LOXORIBINE™ (7-allyl-8-oxoguanosine); LT oral adjuvant (E. coli labile enterotoxin-protoxin); microspheres and microparticles of any composition; MF59™; (squalene-water emulsion); MONTANIDE ISA 51™ (purified incomplete Freund's adjuvant); MONTANIDE ISA 720™ (metabolisable oil adjuvant); MPL™ (3-Q-desacyl-4′-monophosphoryl lipid A); MTP-PE and MTP-PE liposomes ((N-acetyl-L-alanyl-D-isoglutaminyl-L-alanine-2-(1,2-dipalmitoyl-sn-glycero-3-(hydroxyphosphoryloxy))-ethylamide, monosodium salt); MURAMETIDE™ (Nac-Mur-L-Ala-D-GCn-OCH3); MURAPALMITINE™ and D-MURAPALMITINE™ (Nac-Mur-L-Thr-D-isoCln-sn-glyceroldipalmitoyl); NAGO (neuraminidase-galactose oxidase); nanospheres or nanoparticles of any composition; NISVs (non-ionic surfactant vesicles); PLEURAN™ (B-glucan); PLGA, PGA and PLA (homo- and co-polymers of lactic acid and glycolic acid; microspheres/nanospheres); PLURONIC L121™; PMMA (polymethyl methacrylate); PODDS™ (proteinoid microspheres); polyethylene carbamate derivatives; poly-rA: poly-rU (polyadenylic acid-polyuridylic acid complex); polysorbate 80 (Tween 80); protein cochleates (Avanti Polar Lipids, Inc., Alabaster, Ala.); STIMULON™ (QS-21); Quil-A (Quil-A saponin); S-28463 (4-amino-otec-dimethyl-2-ethoxymethyl-1H-imidazo[4,5-c]quinoline-1-ethanol); SAF-1™ (“Syntex adjuvant formulation”); Sendai proteoliposomes and Sendai-containing lipid matrices; Span-85 (sorbitan trioleate); Specol (emulsion of Marcol 52, Span 85 and Tween 85); squalene or Robane® (2,6,10,15,19,23-hexamethyltetracosan and 2,6,10,15,19,23-hexamethyl-2,6,10,14,18,22-tetracosahexane); stearoyltyrosine (octadecyltyrosine hydrochloride); Theramid® (N-acetylglucosaminyl-N-acetylmuramyl-L-Ala-D-isoGlu-L-Ala-dipalmitoxypropylamide); Theronyl-MDP (Termurtide™ or [thr 1]-MDP; N-acetylmuramyl-L-threonyl-D-isoglutamine); Ty particles (Ty-VLPs or virus-like particles); Walter-Reed liposomes (liposomes containing lipid A adsorbed on aluminum hydroxide), and lipopeptides, including Pam3Cys, in particular aluminum salts, such as Adju-phos, Alhydrogel, Rchydragel; emulsions, including CFA, SAF, IFA, MF59, Provax, TiterMax, Montanide, Vaxfectin; copolymers, including Optivax (CRL1005), L121, Poloaxmer4010), etc.; liposomes, including Stealth, cochleates, including BIORAL; plant derived adjuvants, including QS21, Quil A, ISCOMATRIX®, ISCOM; adjuvants suitable for co-stimulation including Tomatine, biopolymers, including PLG, PMM, Inulin; microbe derived adjuvants, including Romurtide, DETOX, MPL, CWS, Mannose, CpG nucleic acid sequences, CpG7909, ligands of human TLR 1-10, ligands of murine TLR 1-13, ISS-1018, IC31, Imidazoquinolines, Ampligen, Ribi529, IMOxine, IRIVs, VLPs, cholera toxin, heat-labile toxin, Pam3Cys, Flagellin, GPI anchor, LNFPIII/Lewis X, antimicrobial peptides, UC-1V150, RSV fusion protein, cdiGMP; and adjuvants suitable as antagonists including CGRP neuropeptide

The compositions may be used for any suitable purpose, including but not limited for delivery of therapeutics. In another embodiment, the gene of interest encodes an antigen, and the compositions may comprise vaccines.

In another embodiment, the disclosure provides pharmaceutical compositions comprising the composition or vaccine of any embodiment herein and a pharmaceutically acceptable carrier.

The disclosure further comprises methods for generating an immune response against an antigen, comprising administering to a subject an amount effective to generate an immune response in the subject of the composition or vaccine of any embodiment herein, wherein the gene of interest encodes an antigen.

The disclosure also provides methods of treating an infection or limiting development of an infection comprising administering to a subject in need thereof the composition or vaccine of any embodiment herein in an effective amount to induce an immune response against the antigen.

In one embodiment, the antigen comprises an HIV antigen, and the subject is at risk of, or has, an HIV infection. In another embodiment, the antigen comprises a SARS-COV-2 antigen, and the subject is at risk of, or has, an SARS-COV-2 infection.

In embodiments where the antigen is an HIV or SARS-COV-2 antigen, “limiting development” includes, but is not limited to accomplishing one or more of the following: (a) generating an immune response (antibody and/or cell-based) HIV or SARS-COV-2 in the subject; (b) generating neutralizing antibodies against HIV or SARS-COV-2 in the subject (b) limiting build-up of HIV or SARS-COV-2 titer in the subject after exposure to HIV or SARS-COV-2; and/or (c) limiting or preventing development of HIV or SARS-COV-2 symptoms after infection. Exemplary symptoms of HIV infection include, but are not limited to, fever, fatigue, swollen lymph nodes, diarrhea, weight loss, oral yeast infection, shingles, and/or pneumonia. Exemplary symptoms of SARS-COV-2 infection include, but are not limited to, fever, fatigue, cough, shortness of breath, chest pressure and/or pain, loss or diminution of the sense of smell, loss or diminution of the sense of taste, and respiratory issues including but not limited to pneumonia, bronchitis, severe acute respiratory syndrome (SARS), and upper and lower respiratory tract infections.

In embodiments where the antigen is an HIV or SARS-COV-2 antigen, “treat” or “treating” includes, but is not limited to accomplishing one or more of the following: (a) reducing HIV titer in the subject; (b) limiting any increase of HIV titer in the subject; (c) reducing the severity of HIV symptoms; (d) limiting or preventing development of HIV symptoms after infection; (e) inhibiting worsening of HIV symptoms; (f) limiting or preventing recurrence of HIV symptoms in subjects that were previously symptomatic for HIV infection; and/or (e) improving survival.

In some embodiments, acceptable formulation materials preferably are nontoxic to recipients at the dosages and concentrations employed. In certain embodiments, the formulation material(s) are for s.c. and/or I.V. administration. In some embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In some embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In certain embodiments, the formulation comprises PBS; 20 mM NaOAC, pH 5.2, 50 mM NaCl; and/or 10 mM NAOAC, pH 5.2, 9% Sucrose. In some embodiments, the optimal pharmaceutical composition will be determined by one skilled in the art depending upon, for example, the intended route of administration, delivery format and desired dosage. See, for example, Remington's Pharmaceutical Sciences, supra. In some embodiments, such compositions may influence the physical state, stability, rate of in vivo release and rate of in vivo clearance of the immunogenic composition.

The subject may be any mammalian subject that may benefit from the methods of the disclosure, including but not limited to humans, dogs, cats, horses, cattle, chickens, goats, etc.

TABLE 1

| Name | Sequence | |
|---|---|---|
| Ifnar | 1 | ggaaggggcg gggcgctgga gggggcgggg cgtcgcggag ggcctagctg cccagaggta |
| (mouse) | 61 | gtctccagct ccgcggtgct gctgaggaga aggaggagaa tgtgagccgc cgcccggcct |
| NCBI | 121 | cccaagacga tgctcgctgt cgtgggcgcg gcggccctgg tgctggtggc cggggcgcct |
| Reference | 181 | tgggtgctac cctcagctgc aggtggagaa aatctgaaac ctcctgagaa tatagacgtc |
| Sequence: | 241 | tacattatag atgacaacta caccctaaag tggagcagcc acggagagtc aatgggcagt |
| NM_010508.2 | 301 | gtgacctttt cagcagaata tcgaacaaaa gacgaggcga agtggttaaa agtgcctgaa |
| SEQ ID | 361 | tgtcaacata ctacaacgac caagtgtgaa ttctctttac tggacacaaa tgtgtatatc |
| NO: 81 | 421 | aaaacacagt ttcgtgtcag agcagaggaa gggaacagca catcttcgtg gaatgaggtt |
| | 481 | gatccgttta ttccattcta cacagctcac atgagccccc cagaagtacg tttagaagct |
| | 541 | gaagataaag ccatactagt ccacatctct cctcccggac aagacgggaa catgtgggca |
| | 601 | ctggagaaac cttccttcag ttacaccata cgaatctggc agaagtcttc cagtgacaaa |
| | 661 | aaaactatta actctacgta ttatgtagaa aagataccag aactcttgcc agagactact |
| | 721 | tactgtttag aagttaaagc aatacatccg tcacttaaga aacacagcaa ttacagcact |
| | 781 | gtgcagtgta taagcaccac agtggcaaat aaaatgcctg tgccaggaaa tctccaagtg |
| | 841 | gatgcccaag gcaagagcta tgtcctgaaa tgggactaca ttgcgtctgc agacgtgctc |
| | 901 | ttcagggcac agtggcttcc tggctattca aaaagcagtt ctggaagccg ttcagataaa |
| | 961 | tggaaaccaa taccaacctg tgcaaatgtc cagactacgc actgtgtctt ttctcaagat |
| | 1021 | actgtctaca caggaacgtt ctttctccat gtacaagcct cagagggaaa tcacacatcc |
| | 1081 | ttttggtctg aagagaagtt tattgattct caaaaacaca ttctccctcc tcctccggtc |
| | 1141 | attactgtca ccgccatgag tgacaccttg cttgtttatg tcaactgtca ggacagcaca |
| | 1201 | tgtgatggac tcaattacga aatcatcttt tgggaaaaca cttccaatac taagataagc |
| | 1261 | atggagaagg atggcccaga gttcaccctc aagaacctgc agccgctgac tgtgtactgt |
| | 1321 | gtccaggcca gagtgctctt cagggccctg ctgaataaga ccagcaactt cagtgaaaag |
| | 1381 | ctgtgtgaga aaacacgtcc aggaagtttt tccacgatct ggattataac tggattaggt |
| | 1441 | gttgtgttct tctctgtcat ggtcctttat gctttgagga gcgtctggaa atacctgtgt |
| | 1501 | catgtgtgct tcccaccact caagcctccc cgcagtattg atgagttttt ctctgagccg |
| | 1561 | ccttcaaaaa accttgtact tctgacggct gaggagcaca cggaaagatg cttcatcatt |
| | 1621 | gagaatacag acacggtcgc tgtagaagta aagcacgcgc ctgaggagga cctcaggaag |
| | 1681 | tacagctcac agaccagcca ggactcgggc aactattcca acgaagagga ggagagtgtg |
| | 1741 | ggcaccgaga gcggccaagc tgtgctctcc aaagctccct gcggggggcc atgcagcgtg |
| | 1801 | cctagccctc ctgggacctt ggaagacggg acctgcttcc tgggaaatga aaaatatctt |
| | 1861 | cagagcccag ccctgaggac agagccagct cttctctgct gacaggtgct cggcccaggc |
| | 1921 | tgggggccgt gtggaggaga cagacttatc tgaagtggct gccagcttct gctgccagct |
| | 1981 | ggaagccctg ggtgctgttt gtgccacaga tgaggctcac tgatgtcagg catctggtgg |
| | 2041 | ttctgtctcg gtgtttgtcc tctaggtgac atttagaaca ttccgatttt taatcctgcc |
| | 2101 | gtagcccctc agtgtgtcac aggtaccatt gtctacagcc gcgcttccta acccatacct |
| | 2161 | caccacagcc gtggcttctg accagaagtc actgaaacca tggagaaagg ggacactaac |
| | 2221 | tggaggagct ggcgttgttt aagatctcag tgtggcctgg gtgtgcccgt tgtactctag |
| | 2281 | tacttgcaga actagaaaat gccagggaca aaaagcaggc atgaaccatt cagtaagaga |
| | 2341 | gaagctagtg aacctgatgg tgaaagaaga ccgtgtccag tcagtgtttg tgtgaaggcc |
| | 2401 | ctgtgctgct ctctgtagcc ttcccgtggt acttataaaa ccaggccgaa ccctgtcctc |
| | 2461 | tgtccggcac tattcaggtc ccttcccagc tccaggtact taaaaaacaa agtgttgggt |
| | 2521 | cctggggtga gggaacagct tcgacggcac agtgctgcct gcaggcgcct ataagtgacc |
| | 2581 | ttagatgaat gctccatgag catcttcggt aggcaggaca tccagcagtg ctggtggctc |
| | 2641 | cagggcagac tcagttgcag gccaaaaagg aagagagagg agtcatagcc ctgccccaat |
| | 2701 | cgagcttgtt gctcgctgca ctgcgccttt gtaatcaatc ttggtttttt tgttttgttt |
| | 2761 | tgtttttttt aacttttagg tgataacata ttctcagata tagcacacaa gagacactac |
| | 2821 | acaaattacg tctgaaagtt ttgatagaaa agagcttttg actggggtag acagcttgct |
| | 2881 | cctcactact accttgtatc tcagtctcct caacagagag agaatgcaga ttgattttca |
| | 2941 | tcctgagttt tattttcttg cccggtttct gggttcgggt gtgctttcaa ggctctgaac |
| | 3001 | agcttgtctg cccacagggg agcccggaga cccagccttt tggctaaatg ttgcaggttc |
| | 3061 | ctgtacttga aggactccag ctcctctgta gagggcatgg tcatcgcggt ctgcaatcac |
| | 3121 | gctgataccc agaaaaatgg ccacacaact caaatctggt gccacgcttt aataattcta |
| | 3181 | cccagcacgg cccagccctg ttcacggcgg aaaagctcct tctgccagga cagcctgtga |
| | 3241 | agaaacaaga cctccgagga cgtggccaag gaaggcccag atgctagcgg gaagttgtta |
| | 3301 | gttggcactc tttcatctct caacaactgt gacacaggtc ttcagggtga agcccaaagc |
| | 3361 | agctatcggt tgcagcagcg ggtaaggagg gggcttcacc tgagcctggc cttctgggcg |
| | 3421 | agcagttcta cctgcacact tcaagacagc ctccatgacc cttttcacgg cacaccgcct |
| | 3481 | gcttttggtg tgaccggggt ttcctgtgca ggcacatgtt cttaaggaat gccatgcaca |
| | 3541 | caggcctgaa gagctggctc agcgggtaga gcacaggttg ctcttgtctg ggatctgagc |
| | 3601 | tctgtcccca gcacccacag agtgcttctg gttgcctcag actccagggg tgcacaaggc |
| | 3661 | catgtaagca aagacatgta agcaaagaca gttgtacaca gagaagtcaa acctttttga |
| | 3721 | aaaaaagaaa caccacgggc tggagagatg gctcagtggt taagaacact gaccactctt |
| | 3781 | ccagaggtcc tgagttcaat tcccagcaac cacatggtgg ctcacaacca tctgtaatag |
| | 3841 | gatctgatgc ccccttctgg tgtgtctgaa gatagctaca gtgtattcat atataaaata |
| | 3901 | ataaatacat cctttttttt ctttttttac tattggatat tttctttatt tacatttcaa |
| | 3961 | atgttatccc ctttcctgat ttacccccct cccagaagcc ccctatccca tcccccctcc |
| | 4021 | cctgcttcta tgagggtgtt cacccaccca cccactccca cttccctgcc cttgattccc |
| | 4081 | ctacactggg ttatttatca agccatcata ggaccaaaga cctctccttc cattgatcca |
| | 4141 | tgacaaagcc atcctctgca acatatgcag cttgagctat gtgtacttct tggttgatgg |
| | 4201 | catagtccct gggagttctg gggcagtggg gggttggggg gagtctggtt agttgatatt |
| | 4261 | attcttccta ttggggttgc aaaccccttc aatgccttca gtcccttctc taactcctct |
| | 4321 | attagggacc ccgagctcag tccagtggtt ggctgctaac atccacctct gtatttgtaa |
| | 4381 | ggctctggca gggcctctca agagacagcc atatcaggct cctttcagca tgtacttctt |
| | 4441 | ggcacccaca acagtgtctg gtttggtaac gctgtacggc atgaattccg aggtgggaca |
| | 4501 | atctatgggt ggcctttcat tcagtctctg ctctacactt tatctccata tttgctccag |
| | 4561 | tgagtatttt gttctccttc taagaaggac agaagcaccc acactttgct cgtccttctt |
| | 4621 | attgagcttc atgtggtctg tgaattgtat cctggttatt tggagctttg gggctagtat |
| | 4681 | ccacttatca gtgagtacat attgtgtgtg ttcttttgtg attgggttac ctcactcagg |
| | 4741 | atgatatcct ccagatccat ccatttgcct aagaatttca tgaattcatc gtttttaata |
| | 4801 | gctgtgcagt actctgttgt ggaaatgcac cacgttttct gtatccattc ctctgttgag |
| | 4861 | gggcatctgg gttctttcca gcttctggct attagaaata aggctgctat gaacatagtg |
| | 4921 | gagcatgtgt ccttcttacc agttggagca tcttctgggt atatgcccag gagtagcata |
| | 4981 | gctgggtcct caggtaatac tatgtccaat tttctgagga ccggccaaac tgatttccag |
| | 5041 | agtggttgta cgagcttgta ttcccaccag caatggagga gtgttcctct ttctccacat |
| | 5101 | cctcaccaac atctgctgtc aacagagttt ttaattttag ccattctgac tggtgtgaaa |
| | 5161 | tggaatctca gggtggtttt gatttgtaat tcaccaatga ctaaggatgt tgaacatttc |
| | 5221 | tttaggtgct tcttggccat ttgatattcc tcagttgaaa attctttgtt tagctctcta |
| | 5281 | ccccattttt taatagggtt atttggttct ctggagtcta acttcttgag ttctttgtat |
| | 5341 | atattggata ttagccctct atcggatgta ggattggtaa agatcttttc ccaatctgtt |
| | 5401 | ggtattgttt tgtccttttg tcagtgtcct ttgccttaca gaagctttgc aattttatga |
| | 5461 | ggtcccactt gtcgattctt tttttttttt tttttttttt tttttcgaga cagggtttct |
| | 5521 | ctgtatagcc ctggctgtcc tggagctcac tttgtagacc aggctggcct cgaactcaga |
| | 5581 | aatccgcctg cctctgcctc ccgagtgctg ggattaaagg cgtgcaccac cacacccggc |
| | 5641 | tccatttgtc gattcttgat cttacagcat tagccattgg tgttctatta aggaattttt |
| | 5701 | tcccctgtac ccgtatgttt gaggctcttt ccaaattgct cctctataaa gagagagagg |
| | 5761 | gggagaggga gaaggagaga gcaaggaagg aagaaaagag aagagacaag acaagaagga |
| | 5821 | aaaggagaga aaagaaaaga gaaaaaagaa atgaaaagaa gagaagagaa aagagaaaaa |
| | 5881 | agaaaagaaa gaccatgcac ttgagggcac tttgtttatg ttgatagttc ttggagaagc |
| | 5941 | tgagcgccgt ctaagattca tgtaaaagcc tcatgagaaa tcagtgcttt cacagacaca |
| | 6001 | tgtaaatagc aacaggcctg tgagcacagc tacacagggg agctggctgc actccactag |
| | 6061 | aattgccacg acgtacctat cacagtgacg tctacagtga gaaatgctgt tctagactca |
| | 6121 | ttacatatgt aatcatttaa atgtaatcat ttagagcctg ggcagaaatc tataacctat |
| | 6181 | gaaaatgttc atttaaaatc aggtcaggca caagtgtgcc tggcttgttc tcatcccagc |
| | 6241 | tgcctaggaa tgtgggccaa cctcagccac atagcaagag cctgtcgtac acatatgtac |
| | 6301 | aggtgtagag gtatgtgtgc atgtatatag gtactatata aaatacagag cccagagcct |
| | 6361 | gtgatgcttc agatggcctc ggacttctct ggagacctgg ggaacgtgtt aacaaagcct |
| | 6421 | tccctaaccc catggaagcc gtgtgtccct gtgtgcattg gtcctttggt gtttgcctcc |
| | 6481 | ctctgtattg cagaagctct ttgagcaggg tctcagttcc tccctgtggc cccccagccc |
| | 6541 | ctgtgacagt gcagcgctgg cacagacagt gcccacagct accgaatgaa tgagatctga |
| | 6601 | aattgaagca gatgactgtt ctggctgttt gtgtaatgaa ataaccacgt ttgtgtgttt |
| | 6661 | ctatgcatca cctttgtgtc tgcatccttt tatatttttg tctggagccc cactgttgta |
| | 6721 | tatcgacaaa cctaacacac accggaacca ggactctgga tctcaggtgc tgctccttta |
| | 6781 | aactggaatc cgtgaggcag aggtgccgtc tacaacccgt tcctgacagc aggacttgca |
| | 6841 | ttctggcatt ctggatccac actgctgtcc cttccaagtg ttactgtctc tcattcctct |
| | 6901 | gtctctgaga tcccttctgc tctacctcaa gagactaaga gggtgaacac agtttgtact |
| | 6961 | tttcatcttc cttacagaga aggaaataat aaattcatat ttgttcccaa gaaaaaaaaa |
| | 7021 | aaaaa |
| Ifnar | 1 | ttaggacggg gcgatggcgg ctgagaggag ctgcgcgtgc gcgaacatgt aactggtggg |
| (human) | 61 | atctgcggcg gctcccagat gatggtcgtc ctcctgggcg cgacgaccct agtgctcgtc |
| SEQ ID | 121 | gccgtgggcc catgggtgtt gtccgcagcc gcaggtggaa aaaatctaaa atctcctcaa |
| NO: 82 | 181 | aaagtagagg tcgacatcat agatgacaac tttatcctga ggtggaacag gagcgatgag |
| | 241 | tctgtcggga atgtgacttt ttcattcgat tatcaaaaaa ctgggatgga taattggata |
| | 301 | aaattgtctg ggtgtcagaa tattactagt accaaatgca acttttcttc actcaagctg |
| | 361 | aatgtttatg aagaaattaa attgcgtata agagcagaaa aagaaaacac ttcttcatgg |
| | 421 | tatgaggttg actcatttac accatttcgc aaagctcaga ttggtcctcc agaagtacat |
| | 481 | ttagaagctg aagataaggc aatagtgata cacatctctc ctggaacaaa agatagtgtt |
| | 541 | atgtgggctt tggatggttt aagctttaca tatagcttac ttatctggaa aaactcttca |
| | 601 | ggtgtagaag aaaggattga aaatatttat tccagacata aaatttataa actctcacca |
| | 661 | gagactactt attgtctaaa agttaaagca gcactactta cgtcatggaa aattggtgtc |
| | 721 | tatagtccag tacattgtat aaagaccaca gttgaaaatg aactacctcc accagaaaat |
| | 781 | atagaagtca gtgtccaaaa tcagaactat gttcttaaat gggattatac atatgcaaac |
| | 841 | atgacctttc aagttcagtg gctccacgcc tttttaaaaa ggaatcctgg aaaccatttg |
| | 901 | tataaatgga aacaaatacc tgactgtgaa aatgtcaaaa ctacccagtg tgtctttcct |
| | 961 | caaaacgttt tccaaaaagg aatttacctt ctccgcgtac aagcatctga tggaaataac |
| | 1021 | acatcttttt ggtctgaaga gataaagttt gatactgaaa tacaagcttt cctacttcct |
| | 1081 | ccagtcttta acattagatc ccttagtgat tcattccata tctatatcgg tgctccaaaa |
| | 1141 | cagtctggaa acacgcctgt gatccaggat tatccactga tttatgaaat tattttttgg |
| | 1201 | gaaaacactt caaatgctga gagaaaaatt atcgagaaaa aaactgatgt tacagttcct |
| | 1261 | aatttgaaac cactgactgt atattgtgtg aaagccagag cacacaccat ggatgaaaag |
| | 1321 | ctgaataaaa gcagtgtttt tagtgacgct gtatgtgaga aaacaaaacc aggaaatacc |
| | 1381 | tctaaaattt ggcttatagt tggaatttgt attgcattat ttgctctccc gtttgtcatt |
| | 1441 | tatgctgcga aagtcttctt gagatgcatc aattatgtct tctttccatc acttaaacct |
| | 1501 | tcttccagta tagatgagta tttctctgaa cagccattga agaatcttct gctttcaact |
| | 1561 | tctgaggaac aaatcgaaaa atgtttcata attgaaaata taagcacaat tgctacagta |
| | 1621 | gaagaaacta atcaaactga tgaagatcat aaaaaataca gttcccaaac tagccaagat |
| | 1681 | tcaggaaatt attctaatga agatgaaagc gaaagtaaaa caagtgaaga actacagcag |
| | 1741 | gactttgtat gaccagaaat gaactgtgtc aagtataagg tttttcagca ggagttacac |
| | 1801 | tgggagcctg aggtcctcac cttcctctca gtaactacag agaggacgtt tcctgtttag |
| | 1861 | ggaaagaaaa aacatcttca gatcataggt cctaaaaata cgggcaagct cttaactatt |
| | 1921 | taaaaatgaa attacaggcc cgggcacggt ggctcacacc tgtaatccca gcactttggg |
| | 1981 | aggctgaggc aggcagatca tgaggtcaag agatcgagac cagcctggcc aacgtggtga |
| | 2041 | aaccccatct ctactaaaaa tacaaaaatt agccgggtag taggtaggcg cgcgcctgtt |
| | 2101 | gtcttagcta ctcaggaggc tgaggcagga gaatcgcttg aaaacaggag gtggaggttg |
| | 2161 | cagtgagccg agatcacgcc actgcactcc agcctggtga cagcgtgaga ctctttaaaa |
| | 2221 | aaagaaatta aaagagttga gacaaacgtt tcctacattc ttttccatgt gtaaaatcat |
| | 2281 | gaaaaagcct gtcaccggac ttgcattgga tgagatgagt cagaccaaaa cagtggccac |
| | 2341 | ccgtcttcct cctgtgagcc taagtgcagc cgtgctagct gcgcaccgtg gctaaggatg |
| | 2401 | acgtctgtgt tcctgtccat cactgatgct gctggctact gcatgtgcca cacctgtctg |
| | 2461 | ttcgccattc ctaacattct gtttcattct tcctcgggag atatttcaaa catttggtct |
| | 2521 | tttcttttaa cactgagggt aggcccttag gaaatttatt taggaaagtc tgaacacgtt |
| | 2581 | atcacttggt tttctggaaa gtagcttacc ctagaaaaca gctgcaaatg ccagaaagat |
| | 2641 | gatccctaaa aatgttgagg gacttctgtt cattcatccc gagaacattg gcttccacat |
| | 2701 | cacagtatct acccttacat ggtttaggat taaagccagg caatctttta ctatg |

### Examples

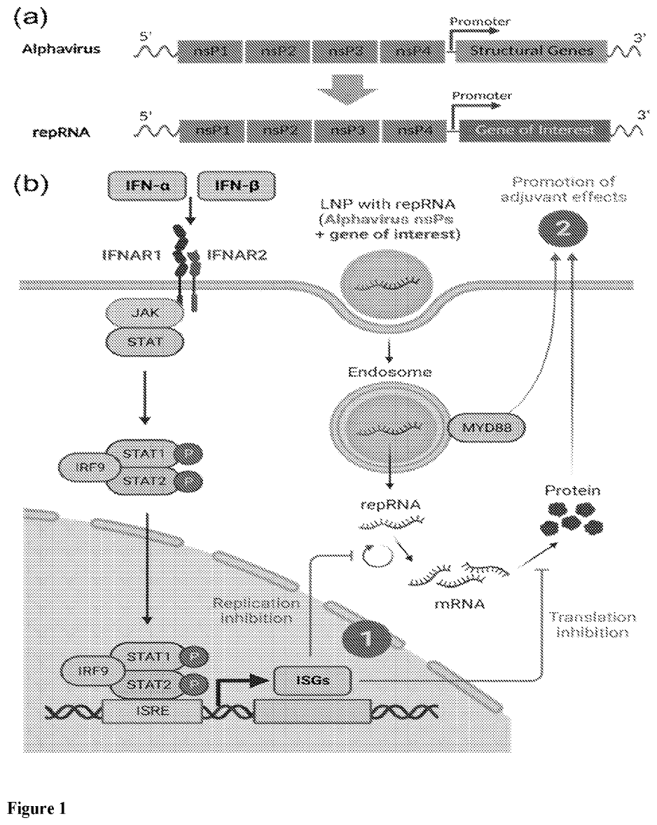

RNA vaccines have emerged as a breakthrough technology in the COVID-19 pandemic. An advancement on this technology uses self-replicating RNA (repRNA) in lieu of mRNAs. Compared to the traditional mRNA, repRNA has the potential for much more prolonged expression, which could be effective for promoting humoral immunity to vaccine immunogens. Thus, we studied the role of innate immune response in repRNA vaccine-elicited immunity. In particular, type 1 interferon (IFN) is a key downstream product from the numerous innate sensors involved in repRNA-recognition. This recognition machinery is particularly important to study for repRNAs, because the repRNA is inherently based on the alphaviral RNA, in which the non-structural protein (nsP) segments of the back-bone enable replication of the gene of interest (FIG. 1a). The segment also allows the repRNA construct to be recognized as a viral infection via activation of toll-like receptor-7 (TLR7)-induced response. This innate immune recognition may hinder the response by inhibiting RNA replication and translation, or by overstimulating innate immunity (FIG. 1b). We thus aimed to test the role of one key downstream component of innate repRNA recognition, the type I interferon response. To this end, we designed repRNA-loaded lipid nanoparticle (LNP) vaccines to express an HIV immunogen (SOSIP trimer of the HIV envelope glycoprotein spike) and silence IFN-α/B receptor 1 (Ifnar1) to suppress type 1 interferon response.

### Results

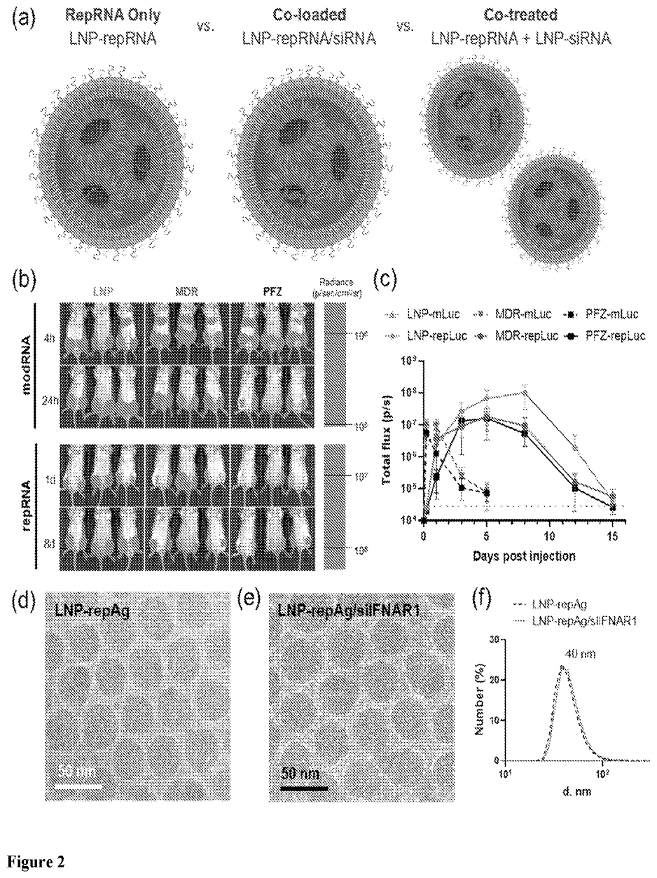

The study aims to examine the role of type I interferon in repRNA recognition. Three lipid nanoparticle (LNP)-formulated repRNA vaccines are compared (FIG. 2a): (1) a baseline LNP that loaded repRNA encoding for the antigen or a reporter; (2) an LNP that co-loads repRNA encoding for the antigen or a reporter and an siRNA against Ifnar1; and (3) a cocktail mix of LNPs, one loading repRNA encoding either an antigen or reporter, and the other loading the siRNA against Ifnar1. By silencing Ifnar1 to interrupt the type I IFN positive feedback loop, an increase in vaccine response would indicate that type I IFN-mediated recognition hinders vaccine-elicited immunity, while a decrease would suggest that type I IFN stimulates it.

Lipid nanoparticles (LNPs) were synthesized using a microfluidic system mixing an organic phase of lipids in ethanol with aqueous phase repRNA in water to induce self-assembly of LNPs encapsulating the replicon (LNP-RNA). LNPs were prepared using a 2:1 ratio of ionizable lipid amine groups to repRNA phosphates (equivalent to a 2.9:1 ionizable lipid:RNA mass ratio). The resulting particles were dialyzed into pH 7.4 phosphate-buffered saline (PBS). Our LNP formulation is composed of N¹,N³,N⁵-tris(3~ (didodecylamino) propyl)benzene-1,3,5-tricarboxamide (TT3), (6Z,9Z,28Z,31Z)-Heptatriaconta-6,9,28,31-tetraen-19-yl 4-(dimethylamino)butanoate (DLin-MC3-DMA), 1,2-dioleoyl-sn-glycero-3-phosphoethanolamine (DOPE), Cholesterol, and 1,2-dimyristoyl-rac-glycero-3-methoxypolyethylene glycol-2000 (DMG-PEG2k) at a molar ratio of 10:25:20:40:5. When loaded with modRNA or repRNA encoding for firefly luciferase and intramuscularly injected into the mouse gastrocnemius, this LNP formulation was able to generate a strong bioluminescence signal that was equivalent to (modRNA), or stronger than (repRNA) clinical benchmark formulations based on Moderna and Pfizer's COVID-19 vaccines (MDR: formulation used for mRNA-123; PFZ: formulation used for BNT162b2) (FIGS. 2b and c). We next characterized the LNPs loaded with either repRNA encoding the immunogen (repAg) alone, or co-loaded with siRNA against Ifnar1 (siIFNAR1). Both formulations form a monodisperse population of spherical nanoparticles that are approximately 40 nm in diameter, as seen in the cryo-EM image in FIGS. 2d and e and confirmed by dynamic light scattering (DLS) measurements shown in FIG. 2f.

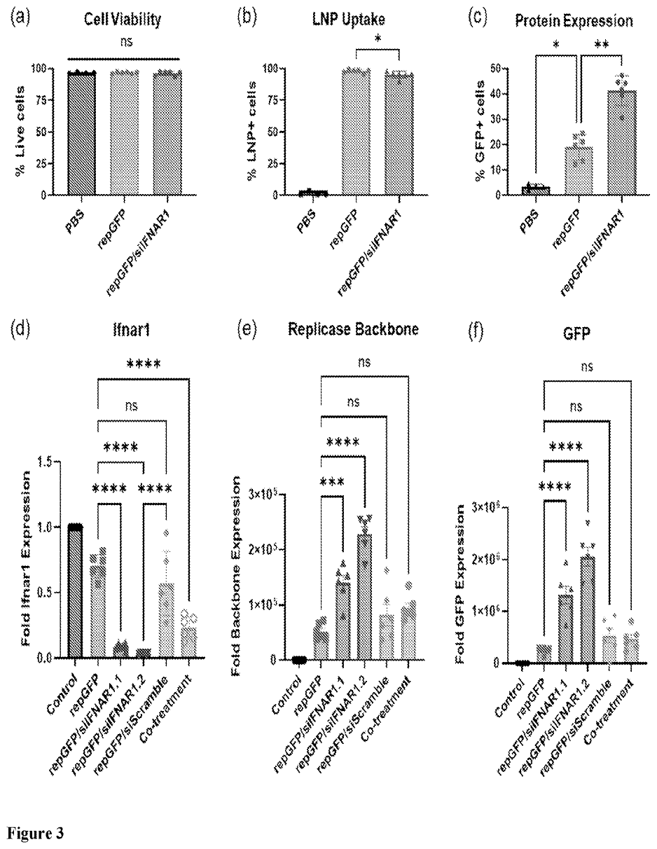

For in vitro evaluation of the impact of Ifnar1 silencing on repRNA transfection, we used C2C12 mouse myoblasts. Flow cytometry analysis revealed that the LNPs loaded with repRNA encoding for GFP (repGFP), or co-loaded with the siRNA against Ifnar1 (siIFNAR1) were non-cytotoxic (FIG. 3a). There was also no significant difference in the uptake amount of the LNPs, regardless of whether the LNPs were loaded with repRNA only or co-loaded with siIFNAR1 (FIG. 3b). Notably, the percentage of GFP-expressing cells doubled from 20% to 40% when we transfected C2C12 cells with the repGFP and siIFNAR1 co-loaded (repGFP/siIFNAR1) LNPs, compared to the standard repGFP-loaded LNPs (FIG. 3c).

Next, we quantified the RNA expression by qRT-PCR. We achieved over 95% knockdown of the Ifnar1 in the groups co-loaded with siRNA (repGFP/siIFNAR1.1 and repGFP/siIFNAR1.2, where siIFNAR1.1 and siIFNAR1.2 indicate two different siRNA sequences against Ifnar1) (FIG. 3d), resulting in a significant increase in the expression of the replicon backbone that encode for the replicase (FIG. 3e), and GFP (FIG. 3f), compared to the standard repGFP-loaded LNPs, as well as those co-loaded with siRNA encoding a scrambled sequence against/fnar1 (repGFP/siScramble) and the co-treated LNPs. Overall, the in vitro results indicate that type I IFN-mediated recognition of LNP-repRNA play an inhibitory role, and that silencing Ifnar1 can enhance transfection efficiency without compromising cell viability or cellular uptake of the nanoparticles.

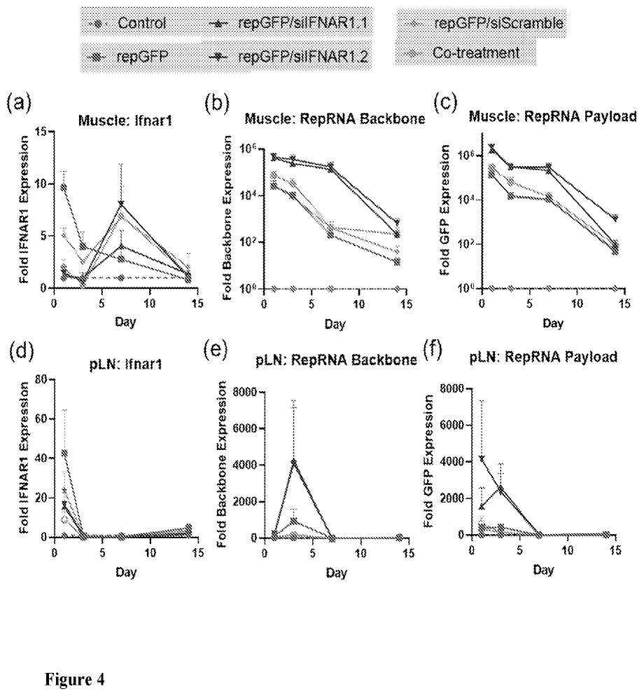

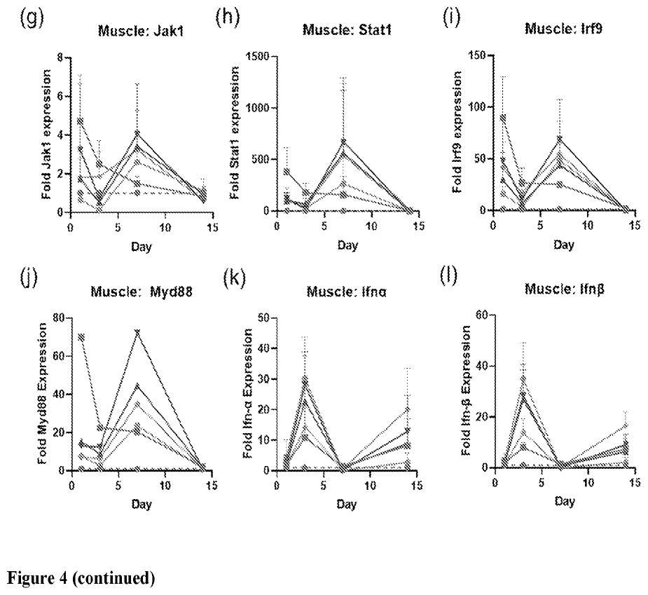

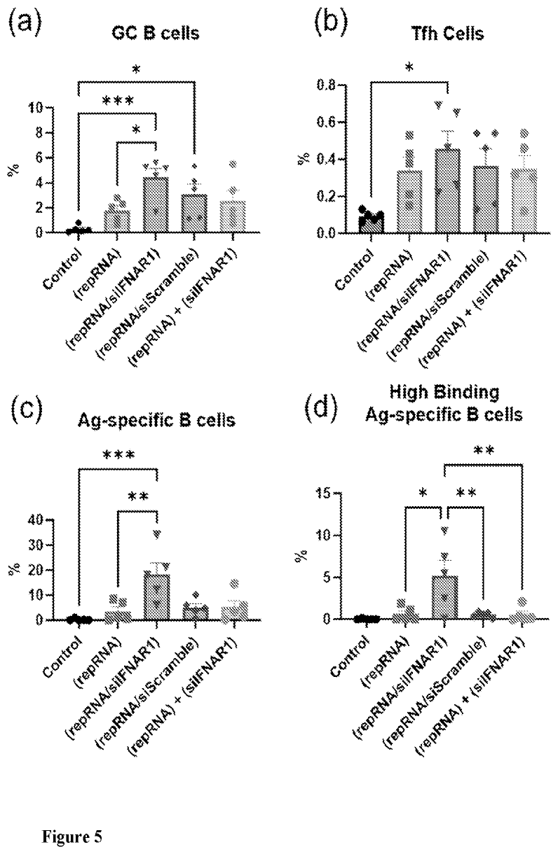

Next, we vaccinated 6-week-old female Balb/c mice intramuscularly with the repGFP-loaded vaccine, the siRNA (siIFNAR1.1, siIFNAR1.2, or siScramble) co-loaded vaccines, or a mixture of the repGFP-loaded and siIFNAR1-loaded LNPs. We purified RNA from the gastrocnemius muscle (injection site), and the draining lymph nodes (popliteal) to quantify RNA expression by qRT-PCR. FIG. 4a-c show the RNA expression levels in the muscle, where we found that LNPs loaded with only the repGFP triggered a strong Ifnar1 expression within 24 h of injection, which rapidly decreased over the following week (FIG. 4a). Conversely, LNPs that were loaded with both repGFP and siINFAR1.1 or siIFNAR1.2, as well as the mixture co-treatment group, effectively silenced Ifnar1 expression for three days following injection, before a sudden recovery at day 7. This led to an approximately 10-fold increase in the expression levels of the repRNA backbone (FIG. 4b) and repRNA Payload (FIG. 4c), GFP, in the siIFNAR1.1 or siIFNAR1.2 co-loaded groups, compared to the standard repGFP vaccine and the siScramble co-loaded vaccine. Interestingly, the co-treatment group displayed only slightly elevated repGFP backbone and GFP expression levels compared to the baseline vaccine, but lower than those in the siIFNAR1.1 and siIFNAR1.2 co-loaded groups, indicating that both the repRNA and siIFNAR1 must be delivered to the same cell to fully enhance repRNA expression.

The primary draining site from the gastrocnemius is the popliteal lymph node (pLN). Here, we found that Ifnar1 expression level was at baseline for all vaccine groups from day 3 post-injection (FIG. 4d). However, the repGFP/siFINAR1.1 and repGFP/siIFNAR1.2 co-loaded groups had elevated expression levels of the repGFP backbone and GFP in the first 3 post-injection, whereas the other vaccine groups had muted expression levels of both genes throughout the 14 days of measurement (FIG. 4e-f). This result indicates a possibility of migratory immune cells being transfected in the gastrocnemius, and trafficking to the popliteal lymph node over the initial 3 days for antigen-presentation. The expression levels of other important markers of the type I IFN signaling pathway—Jak1 (FIG. 4g), Stat1 (FIG. 4h), Irf9 (FIG. 4i), and Myd88 (FIG. 4j)—show similar trend as that of Ifnar1 (FIG. 4a), indicating that knockdown of Ifhar1 shuts down the entire type I IFN pathway downstream of IFNAR1 (JAK1, STAT1, IRF9) as shown in FIG. 1b, as well as interfering with the upstream signal (MYD88) that directly regulates the recognition of endocytosed repRNA. In case of the cytokines, Ifnα (FIG. 4k) and Ifnβ (FIG. 4l), we noted elevated expression levels at day 3 post-vaccination in the Ifhar1-silenced groups, which may have triggered the recovery and boosted expression level of their receptor, Ifnar1, and its downstream signals at day 7 (FIG. 4a, g-i).

To assess how the varying RNA expression levels translated to differences in protein expression, we lysed the gastrocnemius muscles of vaccinated mice to profile anti-viral cytokine levels at days 1, 3, and 7 post-injection (FIG. 4m). At days 1 and 3, the most elevated signal is in the monocyte chemotactic protein-1 (MCP-1), which is a chemokine that attracts monocytes, memory T cells, and dendritic cells to sites of inflammation. Other cytokines that are relatively strongly expressed are interleukin-6 (IL-6), interferon-gamma inducible protein 10 (IP-10), and Regulated on Activation, Normal T-Cell Expressed and Secreted (RANTES), which all stimulate the production of acute phase reactants, activates T cells and B cells, and migrates monocytes. Overall, these cytokines play crucial roles in the recruitment and activation of immune cells in response to tissue damage, infection, and other inflammatory stimuli. As expected, we generally observe diminished expression levels of these cytokines in the repAg/siIFNAR1.1 and repAg/siIFNAR1.2 co-loaded groups that had potently knocked down Ifnar1. Overall, we observe the strongest anti-viral cytokine response in the standard repAg vaccine, followed by the co-treatment group.

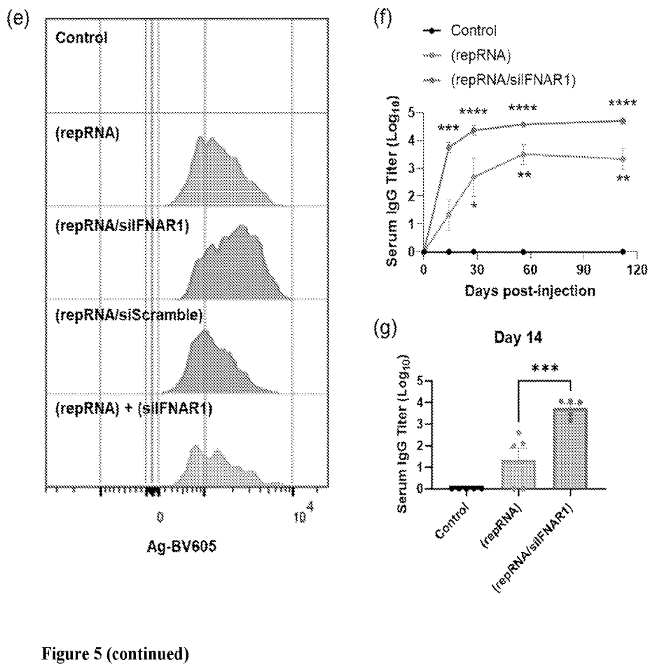

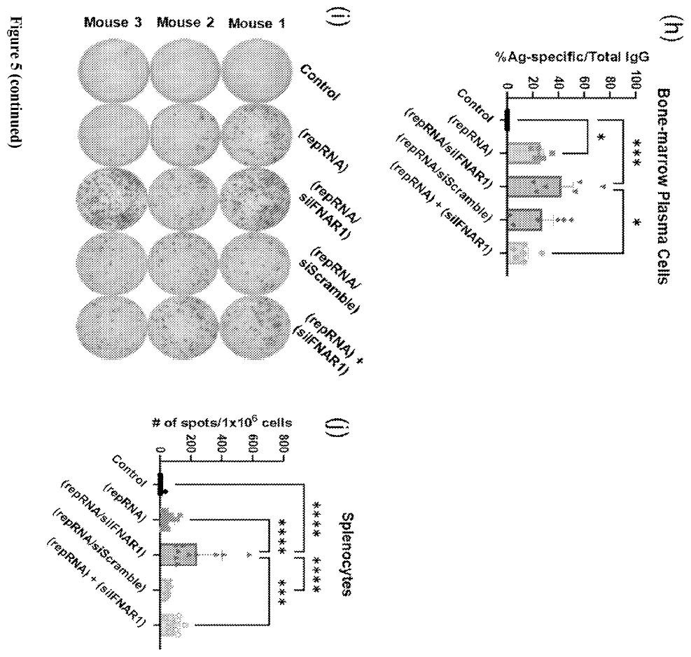

We next assessed how these changes in replicon expression relate to the immunogenicity of LNP-repRNA vaccines. We first immunized mice with LNPs loaded with repRNA encoding N332-GT2 trimer with or without siIFNAR1.2, siScramble, or with the co-treatment vaccine. Flow cytometric analyses of cells recovered from the draining popliteal lymph nodes at day 14 revealed that vaccines co-delivering antigen-encoding repRNA and siIFNAR1 significantly increased expansion of GC B cells, with a trend to increased follicular helper T cells (Tfh, FIG. 5a-b). Most strikingly, siRNA co-delivery amplified trimer-binding GC B cells compared to all of the control groups (FIGS. 5c and c). In particular, the siIFNAR1 co-loaded groups generated significantly more antigen-specific B cells that displayed higher mean fluorescence intensity to both antigen tetramers (FIGS. 5d and e), indicating that these B cells may have higher binding affinity.

A single immunization of balb/C mice with repRNA encoding N332-GT2 HIV Env trimer revealed that co-delivery of replicon and IFNAR-targeting siRNA led to rapid seroconversion, with all animals expressing high titers of trimer-specific IgG by day 14, while mean titers of repRNA alone were ~2 logs lower at this time point (FIG. 5f-g). Antibody titers elicited by repRNA alone slowly rose but even following their plateau at 60 days, they remained 1.4 log lower than titers in the repRNA/siRNA co-delivery groups. To evaluate changes in the development of long-lived plasma cells, ELISPOT analysis of trimer-specific and total IgG-producing cells was carried out on bone marrow plasma cells 6 weeks post vaccination; we compared the ratio between total IgG and HIV immunogen-specific IgG that was produced by the harvested bone-marrow plasma cells at week 6 post-vaccination. This analysis revealed a trend toward increased antigen-specific plasma cell responses elicited by co-delivery of siIFNAR1 with repRNA in the same LNPs (FIG. 5h). Finally, IFN-γ-producing antigen-specific T cell responses in the spleen were also induced significantly more strongly by vaccines that were co-loaded with repAg and siIFNAR1 compared to the baseline repAg vaccine or with a scramble siRNA or the co-treatment group (FIGS. 5i and j).

1. A composition, comprising:
(a) a lipid nanoparticle (LNP);
(b) a self-replicating RNA encoding a gene of interest loaded within the LNP; and
(c) an inhibitory nucleic acid targeting IFN-α/β receptor 1 (Ifnar1) gene loaded within the LNP.

2. The composition of claim 1, wherein the inhibitory nucleic acid comprises a short interfering RNA (siRNA).

3. The composition of claim 1, wherein the inhibitory nucleic acid targets a region of the Ifnar1 gene comprising the nucleotide sequence selected from SEQ ID NO: 1-4, 83, and 89, or selected from SEQ ID NO:1-24 and 83-95.

4. (canceled)

5. The composition of claim 1, wherein the inhibitory nucleic acid comprises a double stranded siRNA sequence comprising the nucleotide sequence of a pair of sequences selected from the following pairs:
(a) sense (5′→3′):
(SEQ ID NO: 25)
GUU GAU CCG UUU AUU CCA UU
and
anti-sense (5′→3′):
(SEQ ID NO: 26)
AAU GGA AUA AAC GGA UCA AC
(IFNAR1.1 mouse minimal sequence);
(b) sense (5′→3′):
(SEQ ID NO: 27)
GUA GAA GUA AAG CAC GCG CC
and
anti-sense (5′→3′):
(SEQ ID NO: 28)
GGC GCG UGC UUU ACU UCU AC
(IFNAR1.2 mouse minimal sequence);
(c) sense (5′→3′):
(SEQ ID NO: 29)
CAA AGC UCA GAU UGG UCC UC
and
anti-sense (5′→3′): anti-sense (5′→3′):
(SEQ ID NO: 30)
GAG GAC CAA UCU GAG CUU UG
(IFNAR1.1 human minimal sequence);
(d) sense (5′→3′):
(SEQ ID NO: 31)
GUG CUC CAA AAC AGU CUG GA
and
anti-sense (5′→3′): (5′→3′):
(SEQ ID NO: 32)
UCC AGA CUG UUU UGG AGC AC
(IFNAR1.2 human minimal sequence);
(e) sense (5′→3′):
(SEQ ID NO: 96)
UCA UAG AUG ACA ACU UUA UC
and
anti-sense (5′→3′):
(SEQ ID NO: 97)
GAU AAA GUU GUC AUC UAU GA
(IFNAR1.3 human minimal sequence);
and
(f) sense (5′→3′):
(SEQ ID NO: 108)
UGU UCA UUC AUC CCG AGA AC
and
anti-sense (5′→3′):
(SEQ ID NO: 109)
GUU CUC GGG AUG AAU GAA CA
(IFNAR1.4 human minimal sequence).

6. The composition of claim 5, wherein the inhibitory nucleic acid comprises a double stranded siRNA sequence comprising the nucleotide sequence of a pair of sequences selected from the following pairs:
(a) sense (5′→3′):
(SEQ ID NO: 25)
GUU GAU CCG UUU AUU CCA UU
and
anti-sense (5′→3′):
(SEQ ID NO: 26)
AAU GGA AUA AAC GGA UCA AC
(IFNAR1.1 mouse minimal sequence);
(b) sense (5′→3′):
(SEQ ID NO: 27)
GUA GAA GUA AAG CAC GCG CC
and
anti-sense (5′→3′):
(SEQ ID NO: 28)
GGC GCG UGC UUU ACU UCU AC
(IFNAR1.2 mouse minimal sequence);
(c) sense (5′→3′):
(SEQ ID NO: 29)
CAA AGC UCA GAU UGG UCC UC
and
anti-sense (5′→3′): anti-sense (5′→3′):
(SEQ ID NO: 30)
GAG GAC CAA UCU GAG CUU UG
(IFNAR1.1 human minimal sequence);
(d) sense (5′→3′):
(SEQ ID NO: 31)
GUG CUC CAA AAC AGU CUG GA
and
anti-sense (5′→3′): (5′→3′):
(SEQ ID NO: 32)
UCC AGA CUG UUU UGG AGC AC
(IFNAR1.2 human minimal sequence);
(e) sense (5′→3′):
(SEQ ID NO: 80)
GUU GAU CCG UUU AUU CCA UUC
and
anti-sense (5′→3′):
(SEQ ID NO: 33)
GAA UGG AAU AAA CGG AUC AAC
(IFNAR1.1 mouse 21 nt sequence);
(f) sense (5′→3′):
(SEQ ID NO: 34)
GUU GAU CCG UUU AUU CCA UUC U
and
anti-sense (5′→3′):
(SEQ ID NO: 35)
AGA AUG GAA UAA ACG GAU CAA C
(IFNAR1.1 mouse 22 nt sequence);
(g) sense (5′→3′):
(SEQ ID NO: 36)
GUU GAU CCG UUU AUU CCA UUC UA
and
anti-sense (5′→3′):
(SEQ ID NO: 37)
UAG AAU GGA AUA AAC GGA UCA AC
(IFNAR1.1 mouse 23 nt sequence);
(h) sense (5′→3′):
(SEQ ID NO: 38)
GUU GAU CCG UUU AUU CCA UUC UAC
and
anti-sense (5′→3′):
(SEQ ID NO: 39)
GUA GAA UGG AAU AAA CGG AUC AAC
(IFNAR1.1 mouse 24 nt sequence);
(i) sense (5′→3′):
(SEQ ID NO: 40)
GUU GAU CCG UUU AUU CCA UUC UAC A
and
anti-sense (5′→3′):
(SEQ ID NO: 41)
UGU AGA AUG GAA UAA ACG GAU CAA C
(IFNAR1.1 mouse 25 nt sequence);
(j) sense (5′→3′):
(SEQ ID NO: 42)
GUA GAA GUA AAG CAC GCG CCU
and
anti-sense (5′→3′):
(SEQ ID NO: 43)
AGG CGC GUG CUU UAC UUC UAC;
(IFNAR1.2 mouse 21 nt sequence);
(k) sense (5′→3′):
(SEQ ID NO: 44)
GUA GAA GUA AAG CAC GCG CCU G
and
anti-sense (5′→3′):
(SEQ ID NO: 45)
CAG GCG CGU GCU UUA CUU CUA C
(IFNAR1.2 mouse 22 nt sequence);
(l) sense (5′→3′):
(SEQ ID NO: 46)
GUA GAA GUA AAG CAC GCG CCU GA
and
anti-sense (5′→3′):
(SEQ ID NO: 47)
UCA GGC GCG UGC UUU ACU UCU AC
(IFNAR1.2 mouse 23 nt sequence);
(m) sense (5′→3′):
(SEQ ID NO: 48)
GUA GAA GUA AAG CAC GCG CCU GAG
and
anti-sense (5′→3′):
(SEQ ID NO: 49)
CUC AGG CGC GUG CUU UAC UUC UAC
(IFNAR1.2 mouse 24 nt sequence);
(n) sense (5′→3′):
(SEQ ID NO: 50)
GUA GAA GUA AAG CAC GCG CCU GAG G
and
anti-sense (5′→3′):
(SEQ ID NO: 51)
CCU CAG GCG CGU GCU UUA CUU CUA C
(IFNAR1.2 mouse 25 nt sequence);
(o) sense (5′→3′):
(SEQ ID NO: 52)
CAA AGC UCA GAU UGG UCC UCC
and
anti-sense (5′→3′):
(SEQ ID NO: 53)
GGA GGA CCA AUC UGA GCU UUG
(IFNAR1.1 human 21 nt sequence);
(p) sense (5′→3′):
(SEQ ID NO: 54)
CAA AGC UCA GAU UGG UCC UCC A
and
anti-sense (5′→3′):
(SEQ ID NO: 55)
UGG AGG ACC AAU CUG AGC UUU G
(IFNAR1.1 human 22 nt sequence);
(q) sense (5′→3′):
(SEQ ID NO: 56)
CAA AGC UCA GAU UGG UCC UCC AG
and
anti-sense (5′→3′):
(SEQ ID NO: 57)
CUG GAG GAC CAA UCU GAG CUU UG
(IFNAR1.1 human 23 nt sequence);
(r) sense (5′→3′):
(SEQ ID NO: 58)
CAA AGC UCA GAU UGG UCC UCC AGA
and
anti-sense (5′→3′):
(SEQ ID NO: 59)
UCU GGA GGA CCA AUC UGA GCU UUG
(IFNAR1.1 human 24 nt sequence);
(s) sense (5′→3′):
(SEQ ID NO: 60)
CAA AGC UCA GAU UGG UCC UCC AGA A
and
anti-sense (5′→3′):
(SEQ ID NO: 61)
UUC UGG AGG ACC AAU CUG AGC UUU G
(IFNAR1.1 human 25 nt sequence);
(t) sense (5′→3′):
(SEQ ID NO: 62)
GUG CUC CAA AAC AGU CUG GAA
and
anti-sense (5′→3′):
(SEQ ID NO: 63)
UUC CAG ACU GUU UUG GAG CAC
(IFNAR1.2 human 21 nt sequence);
(u) sense (5′→3′):
SEQ ID NO: 64)
GUG CUC CAA AAC AGU CUG GAA A
and
anti-sense (5′→3′):
(SEQ ID NO: 65)
UUU CCA GAC UGU UUU GGA GCA C
(IFNAR1.2 human 22 nt sequence);
(v) sense (5′→3′):
(SEQ ID NO: 66)
GUG CUC CAA AAC AGU CUG GAA AC
and
anti-sense (5′→3′):
(SEQ ID NO: 67)
GUU UCC AGA CUG UUU UGG AGC AC
(IFNAR1.2 human 23 nt sequence);
(w) sense (5′→3′):
SEQ ID NO: 68)
GUG CUC CAA AAC AGU CUG GAA ACA
and
anti-sense (5′→3′):
(SEQ ID NO: 69)
UGU UUC CAG ACU GUU UUG GAG CAC
(IFNAR1.2 human 24 nt sequence);
(x) sense (5′→3′):
(SEQ ID NO: 70)
GUG CUC CAA AAC AGU CUG GAA ACA C
and
anti-sense (5′→3′):
(SEQ ID NO: 71)
GUG UUU CCA GAC UGU UUU GGA GCA C
(IFNAR1.2 human 25 nt sequence);
(y) sense (5′→3′):
(SEQ ID NO: 96)
UCA UAG AUG ACA ACU UUA UC
and
anti-sense (5′→3′):
(SEQ ID NO: 97)
GAU AAA GUU GUC AUC UAU GA
(IFNAR1.3 human minimal sequence);
(z) sense (5′→3′):
(SEQ ID NO: 98)
UCA UAG AUG ACA ACU UUA UCC
and
anti-sense (5′→3′):
(SEQ ID NO: 99)
GGA UAA AGU UGU CAU CUA UGA
(IFNAR1.3 human 21 nt sequence);
(aa) sense (5′→3′):
(SEQ ID NO: 100)
UCA UAG AUG ACA ACU UUA UCC U
and
anti-sense (5′→3′):
(SEQ ID NO: 101)
AGG AUA AAG UUG UCA UCU AUG A
(IFNAR1.3 human 22 nt sequence);
(bb) sense (5′→3′):
(SEQ ID NO: 102)
UCA UAG AUG ACA ACU UUA UCC UG
and
anti-sense (5′→3′):
(SEQ ID NO: 103)
CAG GAU AAA GUU GUC AUC UAU GA
(IFNAR1.3 human 23 nt sequence);
(cc) sense (5′→3′):
(SEQ ID NO: 104)
UCA UAG AUG ACA ACU UUA UCC UGA
and
anti-sense (5′→3′):
(SEQ ID NO: 105)
UCA GGA UAA AGU UGU CAU CUA UGA
(IFNAR1.3 human 24 nt sequence);
(dd) sense (5′→3′):
(SEQ ID NO: 106)
UCA UAG AUG ACA ACU UUA UCC UGA G
and
anti-sense (5′→3′):
(SEQ ID NO: 107)
CUC AGG AUA AAG UUG UCA UCU AUG A
(IFNAR1.3 human 25 nt sequence);
(ee) sense (5′→3′):
(SEQ ID NO: 108)
UGU UCA UUC AUC CCG AGA AC
and
anti-sense (5′→3′):
(SEQ ID NO: 109)
GUU CUC GGG AUG AAU GAA CA
(IFNAR1.4 human minimal sequence);
(ff) sense (5′→3′):
(SEQ ID NO: 110)
UGU UCA UUC AUC CCG AGA ACA
and
anti-sense (5′→3′):
(SEQ ID NO: 111)
UGU UCU CGG GAU GAA UGA ACA
(IFNAR1.4 human 21 nt sequence);
(gg) sense (5′→3′):
(SEQ ID NO: 112)
UGU UCA UUC AUC CCG AGA ACA U
and
anti-sense (5′→3′):
(SEQ ID NO: 113)
AUG UUC UCG GGA UGA AUG AAC A
(IFNAR1.4 human 22 nt sequence);
(hh) sense (5′→3′):
(SEQ ID NO: 114)
UGU UCA UUC AUC CCG AGA ACA UU
and
anti-sense (5′→3′):
(SEQ ID NO: 115)
AAU GUU CUC GGG AUG AAU GAA CA
(IFNAR1.4 human 23 nt sequence);
(ii) sense (5′→3′):
(SEQ ID NO: 116)
UGU UCA UUC AUC CCG AGA ACA UUG
and
anti-sense (5′→3′):
(SEQ ID NO: 117)
CAA UGU UCU CGG GAU GAA UGA ACA
(IFNAR1.4 human 24 nt sequence);
and
(jj) sense (5′→3′):
(SEQ ID NO: 118)
UGU UCA UUC AUC CCG AGA ACA UUG G
and
anti-sense (5′→3′):
(SEQ ID NO: 119)
CCA AUG UUC UCG GGA UGA AUG AAC A
(IFNAR1.4 human 25 nt sequence).

7. The composition of claim 5, wherein the 5′ end of the sequences are phosphorylated.

8. The composition of claim 5, wherein the nucleic acids comprise a two nucleotide single stranded overhang at the 3′ terminus, optionally wherein the two nucleotide single stranded overhang at the 3′ terminus comprises dTdT.

9. (canceled)

10. The composition of claim 5, wherein the inhibitory nucleic acid comprises a double stranded siRNA sequence comprising the nucleotide sequence of a pair of sequences selected from the following pairs:
(a) sense (5′→3′):
(SEQ ID NO: 72)
GUU GAU CCG UUU AUU CCA UUC dTdT
and
anti-sense (5′→3′):
(SEQ ID NO: 73)
GAA UGG AAU AAA CGG AUC AAC dTdT
(IFNAR1.1 mouse 21 nt sequence with overhang);
(b) sense (5′→3′):
(SEQ ID NO: 74)
GUA GAA GUA AAG CAC GCG CCU dTdT
and
anti-sense (5′→3′):
(SEQ ID NO: 75)
AGG CGC GUG CUU UAC UUC UAC dTdT;
(IFNAR1.2 mouse 21 nt sequence with overhang);
(c) sense (5′→3′):
(SEQ ID NO: 76)
CAA AGC UCA GAU UGG UCC UCC dTdT
and
anti-sense (5′→3′):
(SEQ ID NO: 77)
GGA GGA CCA AUC UGA GCU UUG dTdT
(IFNAR1.1 human 21 nt sequence with overhang);
and
(d) sense (5′→3′):
(SEQ ID NO: 78)
CAA AGC UCA GAU UGG UCC UCC dTdT
and
anti-sense (5′→3′):
(SEQ ID NO: 79)
GGA GGA CCA AUC UGA GCU UUG dTdT
(IFNAR1.2 human 21 nt sequence with overhang).

11. The composition of claim 10, wherein the 5′ end of the siRNA is phosphorylated, and the 3′ end is hydroxylated.

12. The composition of claim 3, wherein the siRNA comprises one or more locked nucleic acids (LNA).

13. The composition of claim 1, wherein the gene of interest encodes an antigen.

14. The composition of claim 13, wherein the antigen comprises an immunogenic portion of a viral, bacterial, parasitic, protozoan, fungal, or tumor antigen, optionally wherein the antigen comprises a human immunodeficiency virus (HIV) or a severe acute respiratory syndrome (SARS) antigen.

15. (canceled)

16. The composition of claim 1, wherein the LNP comprises an ionizable lipid, a helper lipid, cholesterol, and a polymer-conjugated lipid, optionally wherein the helper lipid comprises phosphocholines, phosphoethanolamines, or combinations thereof.

17. (canceled)

18. The composition of claim 16, wherein the polymer-conjugated lipid comprises polyethylene glycol or polysarcosine conjugated to phosphoethanolamines, 1,2-dimyristoyl-rac-glycero, or other amphiphilic molecules.

19. The composition of claim 16, wherein the amine-to-phosphate (N:P) ratio in the LNP loaded with RNA ranges from about 1:1 to about 20:1.

20. The composition of claim 1, wherein the lipid-to-RNA volume ratio in the LNP ranges from about 1:1 to about 1:20.

21. The composition of claim 1, wherein the LNP comprises
(a) N1,N3,N5-tris(3-(didodecylamino) propyl)benzene-1,3,5-tricarboxamide (TT3),
(b) (6Z,9Z,28Z,31Z)-Heptatriaconta-6,9,28,31-tetraen-19-yl 4-(dimethylamino) butanoate (DLin-MC3-DMA),
(c) 1,2-dioleoyl-sn-glycero-3-phosphoethanolamine (DOPE),
(d) cholesterol, and
(e) 1,2-dimyristoyl-rac-glycero-3-methoxypolyethylene glycol-2000 (DMG-PEG2k);
optionally wherein a molar ratio of LNP components is about 10 (TT3):25 (DLin-MC3-DMA):20 (DOPE):40 (cholesterol):5 (DMG-PEG2k).

22-23. (canceled)

24. A vaccine comprising the composition of claim 13.

25. A pharmaceutical composition comprising the composition of claim 1, and a pharmaceutically acceptable carrier.

26. A method for generating an immune response against an antigen, treating an infection or limiting development of an infection in a subject in need thereof comprising administering to a subject an amount effective to generate an immune response in the subject of the composition of claim 13.

27-29. (canceled)
